(19) United States
(12) Patent Application Publication
(10) Pub. No.: US 2024/0366524 A1
(43) Pub. Date: Nov. 7, 2024

(54) COMPOSITION AND METHOD FOR TREATING OR PREVENTING INFECTIONS

(71) Applicant: CHITOGEL LIMITED, Dunedin (NZ)

(72) Inventor: Peter John Wormald, North Adelaide (AU)

(21) Appl. No.: 18/580,966

(22) PCT Filed: Jul. 22, 2022
(86) PCT No.: PCT/NZ2022/050099

§ 371 Date: Jan. 19, 2024

(30) Foreign Application Priority Data
Jul. 22, 2021 (AU) 2021902255

(51) Int. Cl. A61K 31/047; A61K 9/06; A61K 47/36; A61L 26/00; A61P 31/04

(52) U.S. Cl. CPC A61K 31/047; A61K 9/06; A61K 47/36; A61L 26/0023; A61L 26/008; A61P 31/04; A61L 2300/216

(57) ABSTRACT

The invention relates to a composition and method for treating infections, preventing infections, reducing the likelihood of infections, or reducing the severity of infections in the sinonasal tract, by topically administering a composition comprising glycerol. The invention also relates to a method for supporting a healthy sinonasal microbiome, comprising topically administering a composition comprising glycerol. The invention also relates to a method for promoting the growth of commensal bacteria and inhibiting the growth of pathogenic bacteria in the sinonasal tract, comprising topically administering a composition comprising glycerol.

### FIELD OF THE INVENTION

The invention relates to a composition and method for treating infections, preventing infections, reducing the likelihood of infections, or reducing the severity of infections in the sinonasal tract, by topically administering a composition comprising glycerol. The invention also relates to a method for supporting a healthy sinonasal microbiome, comprising topically administering a composition comprising glycerol. The invention also relates to a method for promoting the growth of commensal bacteria and inhibiting the growth of pathogenic bacteria in the sinonasal tract, comprising topically administering a composition comprising glycerol.

### BACKGROUND

There are ten times more bacterial cells on and in human bodies than there are human cells. Much of the human microbiome is in the digestive system, but the skin and mucosal surfaces are more recently being understood to host an abundant and diverse set of bacteria, fungi and viruses. Microbial communities on healthy skin and mucosal surfaces are predominantly commensal, wherein the host environment supports the growth of the microbiota and the microbiota have either a beneficial effect, or provide no observable effect, on the host.

Studies of the microbiomes of the skin show a prevalence and abundance of microbiota, which varies depending on the skin physiology. Sebaceous, moist and dry areas of skin have significantly different prevalence and abundance of bacterial species, for example. The microbiomes of other parts of the body, such as the sinonasal tract, the middle ear, and the eustachian tube, are all different again.

The effect (beneficial or otherwise) of particular bacteria on the host differs depending on the physiology that they colonise. Bacterial species that are commensal in some physiologies may be pathogenic if they colonise an area with a different physiology. Conversely, bacterial species may be nonviable or have difficulty maintaining a presence on a different physiology.

The diversity of bacterial species in a healthy microbiome will still comprise a number of bacteria associated with disease, but in relatively small numbers. Bacteria that are considered pathogenic can exist in a microbiome without causing disease when they are kept under control by the influence of other bacteria. Pathology often develops only when there is an overgrowth of a particular species resulting in imbalance of a normally balanced microbial community. The presence of commensal bacteria controls the activity and pathogenicity of the bacteria associated with disease. A healthy microbiome is believed to reduce the likelihood of infection by pathogens via the skin or mucosal surface. An abundance of commensal bacteria on the skin and mucosal surfaces reduces the opportunity for pathogenic bacteria to populate.

Commensal bacterial communities are believed to provide a benefit to humans by preventing the overgrowth of pathogens. This effect, known as bacterial interference, occurs due to the antagonism or competition between bacterial species during the process of surface colonisation and acquisition of nutrients. Commensal microbiota may have anti-pathogenic activity via direct competition with pathogens for resources and space for colonisation and/or via their excretion of compounds that inhibit the growth of the pathogenic species. The excretion of proteins (e.g. bacteriocins), short-chain fatty acids, ethanol, lactic acid, acetic acid, and propionic acid by commensal bacteria, for example, are believed to have an anti-pathogenic effect and therefore assist in the maintenance of a healthy microbiome.

Common commensal bacteria of the skin and mucosal surfaces include species of the Cutibacterium family (e.g. Cutibacterium acnes, formerly Propionibacterium acnes) and species of the Corynebacteriaceae family or Corynebacterium genus (e.g. Corynebacterium accolens). Such bacteria are present on the skin, for example.

The microbiome of the skin and several mucosal surfaces have been studied for their effects on diseases or conditions affecting them. Dysbiosis (a loss of bacterial diversity including a loss of beneficial bacteria and an overgrowth of pathogenic bacteria) is associated with several conditions affecting the skin. Conversely, beneficial microbiota have been found to be useful for improving healing and reducing infections in skin wounds.

Shu et al. (Shu M, Wang Y, Yu J, Kuo S, Coda A, et al. (2013) PLOS ONE 8 (2): e55380) describes the treatment of methicillin resistant Staphylococcus aureus skin infections in mice with a combination of C. acnes and glycerol. Shu suggests that the fermentation products of glycerol by C. acnes, including propionic acid, lactic acid and butyric acid, inhibits the growth of MRSA.

Historically, a healthy sinonasal tract was believed to be substantially sterile, but research is emerging about a rich sinonasal microbiome and the role that dysbiosis plays in diseases of the sinonasal zone, such as chronic rhinosinusitis (CRS). The sinonasal microbiome is believed to affect the incidence and severity of infections in the area. For example, despite appropriate medical and surgical management, recalcitrant disease is an ongoing problem for some patients with CRS. Recolonisation of pathogenic bacteria after surgery has been associated with poorer outcomes and recurrence of disease. Mucosal surfaces in the ear, eye, mouth and respiratory tract have also been shown have their own microbiome, and dysbiosis is believed to play a role in diseases of these areas.

Whilst some research indicates that the administration of probiotics may assist in wound healing or reducing infections, there are risks associated with the intentional administration of live bacteria to an affected area of skin or mucosal surface, including the risk that the “beneficial” bacteria may become pathogenic. It is more desirable to promote or support the commensal bacteria already present in a patient's microbiome to interfere with potential pathogens. Accordingly, the inventors believe it is more desirable to administer a composition that preferentially supports the development and maintenance of a patient's own microbiome.

### SUMMARY OF THE INVENTION

The present invention is predicated on the surprising finding of the therapeutic potential in harnessing the sinonasal microbiota for controlling pathogenicity, the treatment of conditions and disorders in the sinonasal tract, and the reduction in likelihood and severity in infections thereof. It has been surprisingly found that compositions comprising glycerol are useful in supporting commensal bacteria, inhibiting pathogenic bacteria, and reducing or preventing infections in the sinonasal tract. Without wishing to be bound by theory, it is believed that the beneficial clinical outcomes described herein following treatment with glycerol compositions are due to the support of a healthy sinonasal microbiome. It is particularly surprising that the support of the sinonasal microbiome was achieved without the application, addition or other inoculation of the sinonasal tract with any microbial strain.

In one aspect, there is provided a method for treating, preventing, reducing the likelihood of, or reducing the severity of, an infection in the sinonasal tract, comprising the topical administration of a composition comprising glycerol to a surface of the sinonasal tract in a subject in need thereof.

The composition may further comprise a hydrogel, preferably a dissolvable hydrogel. Whilst any hydrogel suitable for carrying glycerol and suitable for topical application to a subject would be suitable, the hydrogel preferably comprises a polymer comprising chitosan, such as a dicarboxy-derivatised chitosan polymer. The dicarboxy-derivatised chitosan polymer may be a N-succinyl chitosan polymer. An example of a chitosan polymer preferred for the composition includes a dicarboxy-derivatised chitosan polymer cross-linked to an aldehyde-derivatised dextran polymer.

The composition may comprise glycerol in an amount of at least 15% (v/v). The composition may comprise at least 20% glycerol (v/v). Preferably, the composition may comprise about 20% glycerol (v/v). However, the composition may comprise up to about 90%, 95%, or 100% glycerol (v/v). For example, the glycerol content may be about 15%, 20%, 25%, 30%, 35%, 40%, 45%, 50%, 55%, 60%, 65%, 70%, 75%, 80%, 85%, 90%, 95% or 100% (v/v).

The composition is preferably free of any microorganisms, especially bacterial organisms, including probiotic bacteria. Preferably, the composition is sterile.

Preferably, the method is performed without the inoculation or intentional application or addition of any microorganisms, especially bacterial organisms, including probiotic bacteria. Preferably, the method is performed aseptically.

The topical administration of the composition is preferably to the mucosal surfaces that line the sinonasal tract.

The topical administration may be to a damaged mucosal surface in the sinonasal tract, such as the site of a surgical wound. In this embodiment, the method may comprise the topical administration of the composition to the site of the surgical wound post-operatively, preferably immediately post-operatively. The damaged skin or damaged mucosal surface may be the site of chronic infection, such as chronic rhinosinusitis. Alternatively, the damaged mucosal surface may be a site of a local infection such as an abscess or rhinosinusitis.

The infection may be a post-operative infection in the sinonasal tract. For example, the composition may be applied topically to the sinonasal tract at or around the side of a surgical wound to prevent or reduce the likelihood of a post-operative infection. The infection may be a S. aureus infection, or a Pseudomonas aeruginosa infection. The infection may be a strain of pathogen that is resistant to antibiotics, such as methicillin-resistant S. aureus. The infection may be associated with chronic rhinosinusitis.

The composition preferably is free of antibiotic, antifungal, antibacterial, or antiviral agents. For example, the composition is preferably free of aminoglycoside antibiotics, ansamycin antibiotics, carbacephem antibiotics, cephalosporin antibiotics, glycopeptide antibiotics, lincosamide antibiotics, lipopeptide antibiotics, macrolide antibiotics, monobactam antibiotics, nitrofuran antibiotics, oxazolidinone antibiotics, penicillin antibiotics, polypeptide antibiotics, quinolone antibiotics, sulfonamide antibiotics, tetracycline antibiotics. For the purposes of this application, antibiotic, antifungal, antibacterial, or antiviral agents are agents that have a primary purpose as an antibiotic, antifungal, antibacterial, or antiviral.

The method may further comprise a surgical procedure on the subject's mucosal membrane in which the surrounding mucosa is conserved prior to the administration of the composition to the subject.

In another aspect, there is provided a method for promoting the growth of one or more commensal bacteria in the sinonasal tract, comprising the topical administration a composition comprising glycerol to a surface of the sinonasal tract in a subject in need thereof.

In another aspect, there is provided a method for promoting the growth of one or more commensal bacteria in the sinonasal tract, comprising the topical administration a composition comprising glycerol to a surface of the sinonasal tract in a subject in need thereof.

The commensal bacteria may include bacteria selected from the group consisting of: bacteria in the Corynebacterium genus (such as C. accolens), and C. acnes.

In another aspect, there is provided a method for inhibiting the growth of one or more pathogenic bacteria on the skin or mucosal surface, comprising the topical administration to the skin or mucosal surface a composition comprising glycerol.

The pathogenic bacteria species may include bacteria selected from the group consisting of: S. aureus and P. aeruginosa.

In another aspect, there is provided a method for promoting the growth of one or more commensal bacteria and inhibiting the growth of one or more pathogenic bacteria in the sinonasal tract, comprising the topical administration a composition comprising glycerol to a surface of the sinonasal tract in a subject in need thereof.

In another aspect, there is provided a method for reducing or preventing dysbiosis and/or increasing eubiosis in the sinonasal tract comprising the topical administration of a composition comprising glycerol to sinonasal tract in a subject in need thereof.

In another aspect, there is provided a composition comprising glycerol for use in the treatment, prevention, reduction in the likelihood of, or reduction in the severity of, an infection in the sinonasal tract, wherein the composition is applied topically to a surface of the sinonasal tract in a subject.

In another aspect, there is provided a composition comprising glycerol for use in the promotion of the growth of one or more commensal bacteria in the sinonasal tract, wherein the composition is applied topically to a surface of the sinonasal tract in a subject.

In another aspect, there is provided a composition comprising glycerol for use in the inhibition of the growth of one or more pathogenic bacteria in the sinonasal tract, wherein the composition is applied topically to a surface of the sinonasal tract in a subject.

In another aspect, there is provided a composition comprising glycerol for use in the promotion of growth of one or more commensal bacteria and inhibition of growth of one or more pathogenic bacteria in the sinonasal tract, wherein the composition is applied topically to a surface of the sinonasal tract in a subject.

In another aspect, there is provided a composition comprising glycerol for use in the reduction or prevention of dysbiosis and/or increase in eubiosis in the sinonasal tract, wherein the composition is applied topically to a surface of the sinonasal tract in a subject.

In another aspect, there is provided a use of a composition comprising glycerol in the manufacture of a medicament for the treatment, prevention, reduction in the likelihood of, or reduction in the severity of, an infection in the sinonasal tract, wherein the composition is applied topically to a surface of the sinonasal tract in a subject.

In another aspect, there is provided a use of a composition comprising glycerol in the manufacture of a medicament for the promotion of the growth of one or more commensal bacteria in the sinonasal tract, wherein the composition is applied topically to a surface of the sinonasal tract in a subject.

In another aspect, there is provided a use of a composition comprising glycerol in the manufacture of a medicament for the inhibition of the growth of one or more pathogenic bacteria in the sinonasal tract, wherein the composition is applied topically to a surface of the sinonasal tract in a subject.

In another aspect, there is provided a use of a composition comprising glycerol in the manufacture of a medicament for the promotion of growth of one or more commensal bacteria and inhibition of growth of one or more pathogenic bacteria in the sinonasal tract, wherein the composition is applied topically to a surface of the sinonasal tract in a subject.

In another aspect, there is provided a use of a composition comprising glycerol in the manufacture of a medicament for the reduction or prevention of dysbiosis and/or increase in eubiosis in the sinonasal tract, wherein the composition is applied topically to a surface of the sinonasal tract in a subject.

In another aspect, there is provided a use of a composition comprising glycerol for the promotion of the growth of one or more commensal bacteria in the sinonasal tract, wherein the composition is applied topically to a surface of the sinonasal tract in a subject.

In another aspect, there is provided a use of a composition comprising glycerol for the inhibition of the growth of one or more pathogenic bacteria in the sinonasal tract, wherein the composition is applied topically to a surface of the sinonasal tract in a subject.

In another aspect, there is provided a use of a composition comprising glycerol for the promotion of growth of one or more commensal bacteria and inhibition of growth of one or more pathogenic bacteria in the sinonasal tract, wherein the composition is applied topically to a surface of the sinonasal tract in a subject.

In another aspect, there is provided a use of a composition comprising glycerol for the reduction or prevention of dysbiosis and/or increase in eubiosis in the sinonasal tract, wherein the composition is applied topically to a surface of the sinonasal tract in a subject.

The subjects in need of the method or composition described herein may include subjects suffering from dysbiosis in the sinonasal tract, subjects suffering from rhinosinusitis or chronic rhinosinusitis, subjects suffering from recalcitrant infections in the sinonasal tract, subjects that have undergone sinonasal surgery and need support for the restoration of their sinonasal microbiome, and/or subjects suffering from bacterial infections in the sinonasal tract that are resistant to antibiotics, such as methicillin-resistant S. aureus.

### BRIEF DESCRIPTION OF THE FIGURES

FIG. 1 shows the endoscopic scores for oedema intraoperatively (0 weeks), at 2, 6 and 12 weeks post-operatively in control and treatment side in the study described in Example 3.

FIG. 2 shows the endoscopic scores for granulation tissue intraoperatively (0 weeks), at 2, 6 and 12 weeks post-operatively in control and treatment side in the study described in Example 3.

FIG. 3 shows the proportion of baseline ostial area maintained after 3 months in control vs treatment sides for frontal sinuses, sphenoid sinuses and maxillary sinuses in the study described in Example 3.

FIG. 4 shows the proportion of the microbiome consisting of Corynebacterium at baseline and 3 months post-operative in control and treated groups in the study described in Example 3.

FIG. 5 shows the proportion of the microbiome consisting of Pseudomonas at baseline and 3 months post-operative in control and treated groups in the study described in Example 3.

FIG. 6 shows the proportion of the microbiome consisting of Cutibacterium (C. acnes) at baseline and 3 months post-operative in control and treated groups in the study described in Example 3.

FIG. 7 shows the relative abundance (%) of microbiota at the time of surgery, 12 weeks post-surgery, and 12 months post-surgery, in control and treated groups in the study described in Example 3.

FIG. 8 shows the effect of 20% glycerol on bacterial planktonic growth after 24 hrs in nutrient broth. Data represent the mean±SEM of each bacterial strain; C. accolens (n=4), Corynebacterium propinquum (n=3), Corynebacterium pseudodiphtheriticum (n=3), Staphylococcus epidermidis (n=4), S. aureus (n=4) and P. aeruginosa (n=4), in the study described in Example 4.

FIG. 9 shows the effect of 20% glycerol on C. accolens planktonic growth after 24 hours, in the study described in Example 4. FIG. 9(A) Nutrient Broth. FIG. 9(B): Brain Heart Infusion.

FIG. 10 shows the effect of 20% glycerol on C. propinquum planktonic growth after 24 hours, in the study described in Example 4. FIG. 10(A): Nutrient Broth. FIG. 10(B): Brain Heart Infusion

FIG. 11 shows the effect of 20% glycerol on C. pseudodiphtheriticum planktonic growth after 24 hours, in the study described in Example 4. FIG. 11(A) Nutrient Broth. FIG. 11(B): Brain Heart Infusion

FIG. 12 shows the effect of 20% glycerol on S. epidermidis planktonic growth after 24 hours, in the study described in Example 4. FIG. 12(A) Nutrient Broth. FIG. 12(B): Brain Heart Infusion

FIG. 13 shows the effect of 20% glycerol on S. aureus planktonic growth after 24 hours, in the study described in Example 4. FIG. 13(A) Nutrient Broth. FIG. 13(B): Brain Heart Infusion

FIG. 14 shows the effect of 20% glycerol on P. aeruginosa planktonic growth after 24 hours, in the study described in Example 4. FIG. 14(A) Nutrient Broth. FIG. 14(B): Brain Heart Infusion

FIG. 15 shows the percent survival of C. acnes after 48 hours of incubation (anaerobic environment) in the study described in Example 5.

FIG. 16 shows the percent survival of S. aureus after 48 hours of incubation (aerobic environment) in the study described in Example 6.

FIG. 17 shows the percent survival of S. aureus after 48 hours of incubation (aerobic environment) in media supplemented with the supernatant of C. acnes in the study described in Example 6.

FIG. 18 shows the percent survival of S. aureus after 48 hours of incubation (aerobic environment) in media supplemented with the supernatant of C. acnes in the study described in Example 6. C. acnes was grown in 20% glycerol-supplemented media before extracting the supernatant for this test.

FIG. 19 shows the percent survival of P. aeruginosa after 48 hours of incubation (aerobic environment) in the study described in Example 6.

FIG. 20 shows the percent survival of P. aeruginosa after 48 hours of incubation (aerobic environment) in media supplemented with the supernatant of C. acnes in the study described in Example 6.

FIG. 21 shows the percent survival of P. aeruginosa after 48 hours of incubation (aerobic environment) in media supplemented with the supernatant of C. acnes in the study described in Example 6. C. acnes was grown in 20% glycerol-supplemented media before extracting the supernatant for this test.

### DETAILED DESCRIPTION

### Definitions

As used herein the term “skin” means the outer layers of tissue covering the body.

As used herein, the term “mucosal surface” includes the mucosal membranes and surfaces that line the eyes, ears, inside the nose, the sinonasal tract, the upper respiratory tract, inside the mouth, lip, vagina, urethral opening and the anus.

The term “sinonasal tract” means the nasal cavity, nasal pharynx and all associated sinuses.

As used herein, “topical administration”, “topical application” and the like, means the administration or application to a body surface, such as administration to the surface of the skin or to a mucosal surface. Topical administration to surfaces of the sinonasal tract means the administration or application to a surface, such as a mucosal surface, of the sinonasal tract. Topical administration includes administration to any bodily surface which is exposed, e.g. after surgery, such as exposed bone, periosteum or perichondrium.

As used herein, the term “commensal bacteria” means bacteria colonising a host that under most conditions do not substantially harm the host. For the purposes of this specification, commensal bacteria include bacteria that provide a benefit to the host.

As used herein, the term “pathogenic bacteria” means bacteria that cause disease in suitable conditions.

As used herein, the term “post-operative infection” means any infection that occurs after an invasive medical operation and that is caused by the operation.

As used herein the term “chitosan” means a linear polysaccharide composed of randomly distributed β-(1,4) linked D-glucosamine and N-acetyl-D-glucosamine. Chitosan can be produced by deacetylation of chitin. Both α- and β-chitosan are suitable for use in the invention. The degree of deacetylation (% DA) influences the solubility and other properties of the chitosan. Commercially available chitosan typically has a degree of deacetylation of between about 50 to 100%. A monomer unit of fully deacetylated chitosan is shown in formula I below.

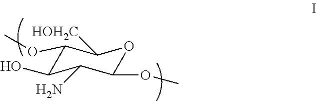

As used herein the term “dicarboxy-derivatised chitosan polymer” means a chitosan polymer that has been derivatised by reaction of a cyclic anhydride with the amine group of some of the D-glucosamine residues of the chitosan polymer. Examples of dicarboxy groups include N-succinyl, N-maloyl and N-phthaloyl. N-succinyl is preferred.

The “dicarboxy-derivatised chitosan polymer” may also be partially derivatised with other functional groups. This secondary derivatisation can occur either at amine positions that are not derivatised with a dicarboxy group or at the hydroxy groups of the D-glucosamine residues. For example, reaction of the cyclic anhydride with an —OH group of the chitosan may lead to some monomers containing ester groups rather than, or in addition to, the amide substituent. If secondary derivatisation is present at the amine position of the dicarboxy-derivatised chitosan polymer, the polymer must retain sufficient free amine groups to be able to form cross-links with the aldehyde-derivatised dextran polymer. Preferably, the dicarboxy-derivatised chitosan polymer is only derivatised by reaction of the cyclic anhydride with the amine group of some of the D-glucosamine residues.

As used herein the terms “N-succinyl chitosan polymer” or “Chitosan N-succinamide” means chitosan that has been derivatised by addition of an N-succinyl group on the amine group of some of the D-glucosamine residues of the chitosan polymer. “N-succinyl chitosan polymer” or “chitosan succinamide” may be used interchangeable. A monomer unit of an N-succinyl chitosan polymer is shown in formula II below.

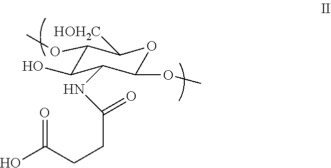

The degree of succinylation may vary. Typically, it is between about 30 to 70%, but the N-succinyl chitosan polymer must retain sufficient free amine groups to be able to form cross-links with the aldehyde-derivatised dextran. The N-succinyl chitosan polymer may also include secondary derivatisation as discussed for the “dicarboxy-derivatised chitosan polymer” (above).

The term “N-succinyl chitosan” as used herein, means an N-succinyl chitosan polymer that is only derivatised with N-succinyl groups at the amine positions and does not include secondary derivatisation with other functional groups.

As used herein the term “dextran” means a glucose polysaccharide composed of α-(1,6) glycosidic linkages with short α-(1,3) side chains. A monomer unit of dextran is shown in formula III below.

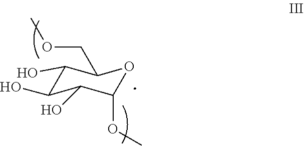

Dextran can be obtained by fermentation of sucrose-containing media by Leuconostoc mesenteroides B512F. Dextrans of molecular weights from 1 kDa to 2000 kDa are commercially available.

As used herein the term “aldehyde-derivatised dextran polymer” means a dextran polymer in which some vicinal secondary alcohol groups have been oxidised to give a reactive bisaldehyde functionality. Aldehyde-derivatised dextran polymers may also be derivatised at other positions with other, functional groups. Preferably, the aldehyde-derivatised dextran polymer is only derivatised at vicinal secondary alcohol groups. A representative monomer unit of aldehyde-derivatised dextran polymer is shown in formula IV below.

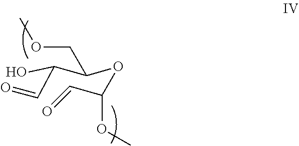

As used herein the term “hydrogel” means a two- or multicomponent system consisting of a three-dimensional network of polymer chains and water that fills the spaces between the macromolecules.

As used herein the term “tissue” means an aggregate of morphologically similar cells with associated intercellular matter that acts together to perform one or more specific functions in the body of an organism including a human. Examples of tissues include but are not limited to muscle, epidermal, nerve and connective tissue.

The term “tissue” also encompasses organs comprising one or more tissue types including but not limited to the chest tissues such as the aorta, the heart, the pleural cavity, the trachea, the lungs, the pericardium and pericardial cavity; the abdominal and retroperitoneal tissues such as the stomach, the small and large intestines, the liver, the pancreas, the gall bladder, the kidneys and the adrenal glands; pelvic cavity tissues including the tissues of the male and female reproductive and urinary tracts; central and peripheral nervous system tissues such as the spinal column and nerves, dura and peripheral nerves; musculoskeletal system tissues such as skeletal muscle, tendons, bones and cartilage; head and neck tissues such as the eye, ear, neck, larynx, nose and paranasal sinuses.

Tissues that are susceptible to adhesion formation are tissues that have been exposed to an inflammatory stimulus. For example, tissues which have been involved in surgical procedures such as but not limited to endoscopic sinus surgery, abdominal surgery, gynaecological surgery, musculoskeletal surgery, ophthalmic surgery, orthopaedic surgery and cardiovascular surgery. Tissues may also be susceptible to adhesion formation following other events such as mechanical injury, disease, for example, pelvic inflammatory disease, radiation treatment and the presence of foreign material, for example, a surgical implant.

As used herein the term “adhesion” means an abnormal attachment between tissues or organs or between tissues and implants that form after an inflammatory stimulus, such as surgery.

The term “granulation” means the growing of new connective tissue and blood vessels on the surface of a wound.

The term “edema” or “oedema” means the accumulation of extracellular fluid. In the case of edema related to surgery, “edema” means swelling that occurs when too much fluid becomes trapped in the tissues of the body, particularly the skin.

As used herein the term “wound” means any damage to a tissue in a living organism including human organisms. The tissue may be an internal tissue such as an internal organ or an external tissue such as the skin. The damage may have resulted from a surgical incision or the unintended application of force to the tissue. Wounds include damage caused by mechanical injuries such as abrasions, lacerations, penetrations and the like, as well as burns and chemical injuries. The damage may also have arisen gradually such as occurs in an ulcer, lesion, sore, or infection. Examples of wounds include, but are not limited to, contused wounds, incised wounds, penetrating wounds, perforating wounds, puncture wounds and subcutaneous wounds.

### Compositions of the Invention

It has been surprisingly found that compositions comprising glycerol promote the growth of commensal bacterial species of the sinonasal microbiome, inhibit the growth of pathogenic bacterial species of the sinonasal microbiome, and treat, prevent, or reduce the incidence and/or severity of infections.

The composition comprising glycerol is adapted for topical administration to skin and mucosal surfaces. The composition may be administered from a syringe or otherwise applied using standard topical administration techniques known in the art.

The composition comprising glycerol is adapted for administration to mucosal surfaces, in particular the mucosal surfaces that line the sinonasal tract. The composition is preferably adapted for administration to areas of the mucosal surface that is damaged, such as by surgery, or by injury or by disease.

The amount of glycerol in the composition may be varied, for example, to optimise the inhibition of particular pathogens and/or promotion of particular commensals. Preferred glycerol content in the composition is about 20% v/v. As shown in Example 4, compositions comprising 20% and 40% (v/v) glycerol inhibited the growth of pathogenic bacteria. It is therefore envisaged that compositions comprising glycerol in an amount between 20% and 40% (v/v) would achieve similar effects. It is further envisaged that compositions comprising somewhat less than 20% (v/v) glycerol, for example 15% (v/v) glycerol, would achieve a similar effect to that observed for 20% (v/v) glycerol. It is therefore envisaged as within the scope of the invention that the glycerol content of the composition may be at least 15% glycerol. For example, the glycerol content may be about 15%, 20%, 25%, 30%, 35%, 40%, 45%, 50%, 55%, 60%, 65%, 70%, 75%, 80%, 85%, 90%, 95% or 100% (v/v). The Examples show that bacterial growth media supplemented with 20% (v/v) glycerol promotes the growth of commensal bacteria and inhibits the growth of pathogenic bacteria. The glycerol content should be balanced to achieve desired promotion of commensals, and inhibition of pathogens. In another Example, the composition comprises greater than 20% (v/v) glycerol, for example 40% (v/v) glycerol.

Preferably, the composition comprises chitosan or a derivative of chitosan. An example of a derivative of chitosan is a dicarboxy-derivatised chitosan polymer cross-linked to an aldehyde-derivatised dextran polymer.

The composition may comprise, or be in the form of, a gel, such as a polymer gel. More preferably, the composition comprises a chitosan-based gel. An example of a chitosan-based gel is one that comprises a dicarboxy-derivatised chitosan polymer cross-linked to an aldehyde-derivatised dextran polymer.

In one example, the composition comprises a dissolvable composition comprising 20% (v/v) glycerol.

Whilst Examples 2 and 3 describe a preferred embodiment of the composition comprising a dissolvable chitosan-based dressing and glycerol, it is envisaged that compositions of the present invention need not comprise a dissolvable dressing of any particular formulation. Examples 4 to 6 show that glycerol added to a bacterial growth media can promote the growth of commensal bacterial species and inhibit the growth of pathogenic bacterial species.

There is no need for the composition to contain any bacteria, such as probiotics or beneficial, or commensal bacteria. The composition is therefore preferably sterile, or prepared under sterile conditions, or applied in a sterile setting.

There is also no need for the composition to contain any antibiotic, antifungal, antibacterial, or antiviral agents. The prevention or reduction in infections achieved by the composition is by supporting commensal bacteria and supporting a healthy microbiome.

The glycerol in the compositions of the present invention is believed to act as a carbon source for commensal bacteria on the mucosal surfaces, especially Corynebacterium spp. and C. acnes. Growth of commensal bacteria is promoted by their consumption and metabolism of the glycerol in the composition, which in turn supports and maintains a healthy microbiome.

### The Polymer Network

In one embodiment, the composition comprises a chitosan-based polymer network. The polymer network is preferably formed by derivatisation and cross-linking of two well-known polymers; chitosan and dextran. The polymer rapidly forms a three-dimensional polymer network, creating a hydrogel in aqueous solution. The properties of the hydrogel can be tailored for specific applications by modifying the derivatisation and cross-linking of the two polymer components.

The dicarboxy-derivatised chitosan component and aldehyde-derivatised dextran component used in the present invention are described in WO2009/028965, the contents of which are incorporated herein by reference.

### The Chitosan Component

Chitosan is widely available and can be obtained commercially from a range of sources, for example, Sigma-Aldrich (www.sigma-aldrich.com).

Alternatively, chitosan can be prepared by deacetylation of chitin. Many deacetylation methods are known in the art, for example, hydrolysing chitin in a concentrated solution of sodium hydroxide on heating and then recovering chitosan by filtering and washing with water. Chitin exists as either α-chitin or β-chitin. Chitin is found in crustaceans, insects, fungi, algae and yeasts, α-chitin is obtained predominantly from the shells of crustaceans such as lobster, crab and shrimp, whereas β-chitin is derived from squid pens. Both types of chitin can be used to prepare the dicarboxy-derivatised chitosan for use in the polymer network.

Generally, the average molecular weight (MWₐᵥ) of commercially available chitosan is between about 1 to 1000 kDa. Low molecular weight chitosan has a MWₐᵥ of about 1 to 50 kDa. High molecular weight chitosan has a MWₐᵥ of about 250 to 800 kDa. Chitosan of any MWₐᵥ can be used in the polymer network.

Deacetylation of chitin means that the resulting chitosan has a majority of free, primary amine groups along its polymeric backbone. The degree of deacetylation of the chitosan may influence the properties of the polymer network because only those glucosamine units that are deacetylated are available for derivatisation or cross-linking. In addition, the solubility of the chitosan depends on the degree of deacetylation.

Chitosan polymers most suitable have a degree of deacetylation of between about 40% to 100%. Preferably, the degree of deacetylation is between about 60% to 95%, more preferably, between about 70% to 95%.

Chitosans for use in the polymer network of the invention are dicarboxy-derivatised at the amine made free by deacetylation of the chitin. Dicarboxy-derivatised chitosan polymers can be made by reacting chitosan with a cyclic acid anhydride. Cyclic acid anhydrides suitable for use in the polymer network of the invention include succinic anhydride, maleic anhydride, phthalic anhydride, glutaric anhydride, citraconic anhydride, methylglutaconic anhydride, methylsuccinic anhydride and the like.

Preferably, the dicarboxy-derivatised chitosan polymer is made from the reaction of chitosan and one or more of succinyl anhydride, phthalic anhydride, or glutaric anhydride. More preferably, the dicarboxy-derivatised chitosan polymer is made from the reaction of chitosan and succinyl anhydride.

Derivatisation can be achieved by any method known in the art. For example, the solid chitosan can be heated in a solution of cyclic anhydride in DMF or solubilised in a methanol/water mixture and then reacted with the anhydride. Other solvents suitable for use in the derivatisation process include dimethylacetamide. Acids such as lactic acid, HCl or acetic acid can be added to improve the solubility of the chitosan. A base such as NaOH is typically added to deacetylate some of the acetylated amine groups.

An exemplary method is provided in WO2009/028965. The method used can be selected depending on the cyclic anhydride used and/or the average molecular weight of the chitosan. Both the chitosan and the cyclic anhydride should be able to substantially dissolve or swell in the solvent used.

In a preferred embodiment, the dicarboxy-derivatised chitosan is N-succinyl chitosan. Methods of preparing N-succinyl chitosan are well known in the art. See for example, “Preparation of N-succinyl chitosan and their physical-chemical properties”, J Pharm Pharmacol. 2006, 58, 1177-1181.

The reaction of the cyclic anhydride with the chitosan acylates some of the free amine positions with dicarboxy groups. For example, when the cyclic anhydride used is succinic anhydride, some of the amine groups are N-succinylated. The NaOH treatment following N-succinylation removes some of the acyl groups from the amine groups in the chitosan. Increasing the temperature of the NaOH treatment increases the percentage of free amine groups present, as demonstrated in WO2009/028965.

The degree of acylation is indicated by the ratio of C:N in the product. The degree of acylation can also be determined by ¹H nuclear magnetic resonance spectroscopy. An N-succinyl chitosan polymer is represented below. Formula V shows the three types of D-glucosamine units present in the polymer—the N-succinylated-D-glucosamine, the free D-glucosamine, and the N-acetyl-D-glucosamine.

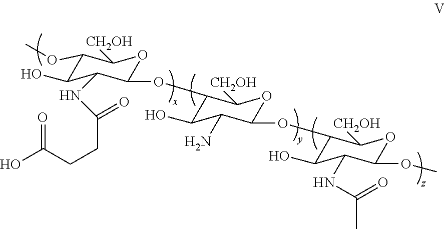

In one embodiment, x is between about 60 to 80%, y is between about 1 to 15% and z is between about 10 to 25%.

In another embodiment, x is between about 60 to 80%, y is between about 1 to 30% and z and between about 2 to 25%.

High degrees of anhydride substitution render the dicarboxy-derivatised chitosan polymer more soluble but may hinder cross-linking to the aldehyde-derivatised dextran polymer.

In one embodiment, the dicarboxy-derivatised chitosan polymer is between about 20% and 80% dicarboxy-derivatised. Preferably, the dicarboxy-derivatised chitosan polymer is between about 30% and 60% dicarboxy-derivatised. More preferably, dicarboxy-derivatised chitosan polymer is between about 45% and 50% dicarboxy-derivatised. In one embodiment, the dicarboxy-derivatised chitosan polymer is between about 50% and 90% dicarboxy-derivatised. Preferably, the dicarboxy-derivatised chitosan polymer is between about 60% and 80% dicarboxy-derivatised.

### The Dextran Component

Dextran is a polysaccharide made of D-glucose units linked predominantly by α-1,6 linkages. Crude, high molecular weight dextran is commercially obtained by growing L. mesenteroides on sucrose. The resulting polysaccharide is hydrolysed to yield low molecular weight dextrans.

Before dextran can be cross-linked to the dicarboxy-derivatised chitosan polymer, it must be activated. Reactive bisaldehyde functionalities can be generated from the vicinal secondary alcohol groups on dextran by oxidation. Typical methods are provided in WO2009/028965. The resulting aldehyde-derivatised dextran polymer can then be reductively coupled to the primary amine groups of the dicarboxy-derivatised chitosan to form a cross-linked polymer network of the invention.

In one embodiment, the oxidising agent is sodium periodate. Other suitable oxidising agents include potassium periodate and the like.

The oxidised product, the aldehyde-derivatised dextran polymer, actually only contains a small amount of free aldehyde groups. Most of the aldehyde groups are masked as acetals and hemiacetals, which are in equilibrium with the free aldehyde form of the dextran. Reaction of some of the free aldehyde groups causes the equilibrium to shift from the acetal and hemiacetal form, towards the formation of more free aldehyde groups.

The degree of oxidation can be influenced by the molar ratio of the oxidising agent used. A higher degree of oxidation will provide an aldehyde-derivatised dextran polymer with more sites available for cross-linking. However, a lower degree of oxidation will result in a more soluble aldehyde-derivatised dextran polymer. The periodate reaction also dramatically decreases the molecular weight of the dextran polymer.

In one embodiment, the degree of oxidation is between about 30% to about 100%, more preferably between about 50% to about 100%. Most preferably, the degree of oxidation is between about 80 to about 100%. WO2009/028965 compares gelling times for polymer networks of the invention prepared using aldehyde-derivatised dextran polymers with different degrees of aldehyde-derivatisation (or oxidation). More highly aldehyde-derivatised dextran polymers have lower molecular weights and form gels faster, when combined in solution with solutions of N-succinyl chitosan.

The degree of derivatisation can be measured using the extended reaction with hydroxylamine hydrochloride and then titration of the liberated protons (Zhao, Huiru, Heindel, Ned D, “Determination of degree of substitution of formyl groups in polyaldehyde dextran by the hydroxylamine hydrochloride method,” Pharmaceutical Research (1991), 8, page 400-401).

It has been found that the aldehyde groups of the aldehyde-derivatised dextran polymer are susceptible to react with nearby hydroxyl groups, forming hemiacetals or hemialdals, in water. Aldehyde-derivatised dextran polymer will therefore degrade over time in an aqueous solution, causing to shorter viable shelf life. Therefore, it has been found that the aldehyde-derivatised dextran polymer should be stored as a solid to maximise its shelf life, particularly beyond 12 months from manufacture.

Cross-Linking the Chitosan Component with the Dextran Component

The polymer network comprises a dicarboxy-derivatised chitosan polymer cross-linked to an aldehyde-derivatised dextran polymer. In one embodiment the dicarboxy-derivatised chitosan polymer is an N-succinyl chitosan polymer. In one embodiment the N-succinyl chitosan polymer is cross-linked to the aldehyde-derivatised dextran polymer through the amine group of the N-succinyl chitosan polymer and the aldehyde group of the aldehyde-derivatised dextran polymer. Preferably, the N-succinyl chitosan polymer is N-succinyl chitosan.

To make the polymer network, the dicarboxy-derivatised chitosan polymer is cross-linked to the aldehyde-derivatised dextran polymer. This can be achieved by mixing aqueous solutions of the two polymers. For example, see WO2009/028965.

In one embodiment, it is desirable that the aqueous solution in which the polymer matrix forms, has a pH of about 6 to 8, preferably between about 6.5 to 7.5. This can be achieved by adjusting the pH of the separate aqueous solutions of the polymer components to within this range before mixing the two solutions. Alternatively, the pH of the aqueous solutions of the individual polymer components can be adjusted following dialysis, prior to freeze drying. The pH can be adjusted using any suitable base or acid. Generally, the pH will be adjusted using NaOH.

In one embodiment either or both aqueous solutions may independently contain one or more pharmaceutically acceptable excipients. In one embodiment the aqueous solutions may independently contain NaCl. Preferably, the concentration of NaCl is between about 0.5 to 5% w/v. More preferably, the concentration of NaCl is between about 0.5% to 2% w/v, most preferably about 0.9% w/v.

In one embodiment the aqueous solutions may independently contain one or more buffers including but not limited to phosphate buffers such as sodium phosphate (e.g. Na₂HPO₄), acetate buffers, carbonate buffers, lactate buffers, citrate buffers and bicarbonate buffers.

### The Polymer Network Hydrogels

The dicarboxy-derivatised chitosan polymer reacts with the aldehyde-derivative dextran polymer, to produce a three-dimensional cross-linked polymer network. This polymer network forms a hydrogel with the aqueous solution in which it is formed. The hydrogel of the invention has properties that make it suitable for use in medicinal applications, in particular, wound healing, prevention of surgical adhesions, and reducing bleeding (haemostasis).

Without wishing to be bound by theory, it is believed that application of the hydrogel of the invention to a wound surface prevents the formation of fibrin and blood clots within this space thereby preventing subsequent formation of adhesions.

The properties of the hydrogel can be tailored for specific applications by modifying the derivatisation and cross-linking of the two polymers.

In the polymer networks of the invention, the amine groups of the D-glucosamine residues of chitosan may be
(a) cross-linked to the aldehyde-derivatised dextran polymer,
(b) acylated with a dicarboxy group, or
(c) acetylated (from the original chitin material).

High degrees of acetylation and/or dicarboxy acylation will leave fewer free amine groups to cross-link with the aldehyde-derivatised dextran polymer. Consequently, when the aqueous solutions of the two polymers are mixed, the amount of polymerisation that occurs will be affected by the acylation and acetylation patterns of the dicarboxy-derivatised chitosan polymer. This in turn will affect how quickly, if at all, the hydrogel is formed. If very little polymerisation occurs in a dilute solution of the polymers, no hydrogel will be formed.

The aqueous solutions of dicarboxy-derivatised chitosan polymer and aldehyde-derivatised dextran polymer comprise between about 2% to about 10% w/v of each component.

Generally, aqueous solutions of equal concentrations of the two polymers are mixed to form the hydrogel of the invention. However, different ratios of dicarboxy-derivatised chitosan polymer and aldehyde-derivatised dextran polymer can be used, provided the properties of the two polymers are such that they cross-link to form a hydrogel of the invention when mixed.

A person skilled in the art can manipulate the parameters of
(a) degree of deacetylation of chitosan,
(b) degree of dicarboxy-derivatisation of chitosan,
(c) degree of oxidation of aldehyde-derivatised dextran, and
(d) concentration in aqueous solution,
so that the component polymer solutions rapidly cross-link to form a hydrogel when mixed.

### Preparation of a Dissolvable Composition

In one example, the dissolvable composition may be prepared by combining the aldehyde-derivatised dextran polymer and the N-succinyl chitosan polymer in a solvent comprising water and glycerol. The solvent may comprise an aqueous buffer and glycerol, preferably under aseptic conditions. The glycerol is present in an amount of about 20% (v/v). An example of the method of preparation of the dissolvable composition is described in Example 2.

### Administration of the Dissolvable Composition to a Subject

Once prepared, the dissolvable composition may be applied topically to the sinonasal tract. In Example 3, the dissolvable composition is applied to a subject's sinonasal tract following sinus surgery. However, the dissolvable composition may be topically applied to any area of skin or mucosal surface. For example, the dissolvable composition may be applied to the site of a surgical wound, or to areas of damage to skin or mucosal surface, or other areas in which the healthy microbiome has been disrupted.

### Glycerol Composition for Supporting the Microbiome

It has been surprisingly found that compositions comprising glycerol promote the growth of commensal bacterial species and inhibit the growth of pathogenic bacterial species. In particular, the Examples show that compositions comprising about 20% v/v glycerol promote the growth of commensal bacterial species and inhibit the growth of pathogenic bacterial species in the sinonasal tract. The promoted bacterial species commensal to the sinonasal tract include C. accolens, C. propinquum and C. acnes.

Example 3 shows that the sinonasal tract of subjects suffering from chronic rhinosinusitis (CRS) comprises C. acnes and bacteria of the Corynebacterium genus, such as C. accolens and C. propinquum species. Examples 4 and 5 further demonstrate the effect of glycerol on the growth of bacterial isolates. In Example 4, the growth of C. accolens isolated from the sinonasal tract of subjects suffering from CRS in media (Nutrient Broth (NB) and Brain-Heart Infusion (BHI)) comprising 20% v/v glycerol was either unaffected or promoted compared to a control. Similarly, Example 5 shows that C. acnes growth is supported in the presence of glycerol. In contrast, Example 4 shows that growth of other bacterial strains isolated from CRS subjects, including C. propinquum, C. pseudodiphtheriticum, S. epidermidis, S. aureus, and P. aeruginosa, is inhibited in media comprising 20% v/v glycerol compared to control.

Without wishing to be bound by theory, it is believed that the promotion of the growth of C. accolens and C. acnes and simultaneous inhibition of pathogenic bacterial species is one reason for the beneficial post-operative outcomes observed in the subjects treated with the dissolvable composition (comprising 20% v/v glycerol) described in Example 3.

Surprisingly, Example 4 further shows that compositions comprising an amount of glycerol of about 40% (v/v) have a significant inhibitory effect on the growth of bacterial species in the sinonasal microbiome. Cultures of the bacterial isolates in growth media (Nutrient Broth (NB) and Brain-Heart Infusion (BHI)) containing 40% v/v glycerol showed a significant inhibitory effect on the growth of all bacterial species measured, including C. accolens, C. propinquum, C. pseudodiphtheriticum, S. epidermidis, S. aureus and P. aeruginosa.

Whilst the glycerol composition of the present invention may be administered in a post-operative setting to support the recovery of a sinonasal microbiome, the glycerol composition may also be administered to support a healthy microbiome on areas of damaged skin or mucosal surfaces, including damaged skin or mucosal surfaces in which the normal microbiome of that area has been altered or destroyed.

### In Vivo Support of the Microbiome

It has been surprisingly found that the topical administration of a composition comprising glycerol to the sinonasal tract may be used to prevent or inhibit the growth of pathogenic bacteria. The inhibited bacterial species that are pathogenic to the sinonasal tract include C. propinquum, C. pseudodiphtheriticum, S. epidermidis, S. aureus, and P. aeruginosa.

It has further been found that the topical administration of a composition comprising glycerol to the sinonasal tract may be used to promote the growth of commensal bacteria. The promoted bacterial species commensal to the sinonasal tract include C. accolens, C. propinquum, and C. acnes.

Example 3 shows that the post-operative application of a dissolvable composition (containing 20% v/v glycerol) to subjects having undergone sinonasal surgery is assistive in the development of a healthy sinonasal microbiome and inhibits the growth of pathogenic bacteria therein. For example, the dissolvable composition described in Example 2 was found to support the growth of commensal bacteria, such as strains of the Corynebacterium genus and C. acnes, whilst also inhibiting the growth of pathogenic bacteria, such as pathogenic bacteria of the Pseudomonas genus.

Example 3 shows that three months after sinonasal surgery, subjects administered the composition of Example 2 experienced a greater increase in growth of corynebacteria, an increase in the proportion of C. acnes, and a smaller increase in the growth of pathogenic Pseudomonas bacteria, compared to the control side. Three months after sinus surgery, the proportion of corynebacteria in the microbiome increased from 19.1% to 31.7% in the control group, and from 17.0% to 42.1% in the treated group. The proportion of Pseudomonas in the microbiome was 8.7% in treated group compared to 13.7% in the control group. The proportion of the microbiome that consisted of C. acnes decreased from 22.5% to 6.9% in the control group but increased from 10.6% to 13.2% in the treatment group.

The promotion of C. acnes and C. accolens in the sinonasal microbiome by the composition indicates that the composition is useful for maintaining a healthy microbiome of the surfaces in the sinonasal tract.

The surgical process severely damages the microbiome of the local skin or mucosal surface. Post-operative skin and mucosal surfaces are a barren microbial environment. The application of the composition of the invention to areas of surgical procedures promotes the recovery of a healthy microbiome by selectively promoting the growth of commensal bacteria including C. acnes, and bacteria of the Corynebacterium genus such as C. accolens.

### Reducing the Rate of Infections

Example 3 showed that the use of a dissolvable composition comprising 20% v/v glycerol caused a significant decrease in the proportion of post-operative infections. Post-operative infections in surgical sites treated with the dissolvable composition were about 23%, compared to 77% in the control group (p=0.04). The application of the dissolvable composition following a surgical procedure therefore has significant efficacy in reducing the likelihood of a subject contracting a post-operative infection.

Without wishing to be bound by theory, it is believed that the support of a healthy microbiome by the composition of the present invention reduces the likelihood of establishment of pathogens on the skin or mucosal surface. This in turn reduces the likelihood of pathogens overgrowing (proliferating) on the skin and mucosal surfaces and causing infections.

Whilst the composition of the present invention may be administered in a post-operative setting to reduce or prevent post-operative infections, the composition may also be administered to areas of damaged skin or mucosal surface to reduce the likelihood of infections. Examples of infections on the skin that may be reduced or prevented include abscesses and cellulitis, and on a mucosal surface may be chronic sinusitis or chronic suppurative otitis media.

### Combinations of Glycerol and Derivatives of Chitosan

The experiments described in Example 5 show that glycerol can support the growth of C. acnes. Further, Example 5 indicates that a combination of glycerol and a chitosan-dextran derivative, namely dicarboxy-derivatised chitosan cross linked with aldehyde-derivatised dextran (i.e., CS-DA-GLY) also supports the growth of C. acnes. This result is particularly surprising considering the finding that the derivative of chitosan, in the absence of glycerol (i.e., CS-DA), caused a decrease in the growth of C. acnes. Example therefore indicates synergism in the combination of the chitosan-dextran derivative and glycerol in promoting the growth of C. acnes.

Example 6 provides insight into the interrelationship between glycerol, chitosan-dextran derivatives, and commensal bacteria on the inhibition of the growth of pathogenic bacteria. Surprisingly, Example 6 shows that glycerol, even in the absence of other agents, provides a clear inhibitory effect on the growth of S. aureus. An inhibitory effect is also observed when glycerol is combined with the chitosan-dextran derivative (i.e., CS-DA-GLY) for both S. aureus and P. aeruginosa, in the absence of other agents. Example 6 further investigated the role of the commensal bacteria by the addition of the supernatant of C. acnes cultures grown in the presence or absence of glycerol to the pathogens' growth media. The results showed that the supernatant of C. acnes culture grown in the presence of glycerol provided a strong inhibitory effect on the growth of S. aureus and P. aeruginosa, especially when the pathogens were additionally exposed to either glycerol or combinations of glycerol and chitosan-dextran derivatives.

The results and observations of Examples 5 and 6 appear consistent with a hypothesis that the metabolism of glycerol by C. acnes produces agents that inhibit the growth of pathogenic bacteria such as S. aureus and P. aeruginosa. However, Examples 5 and 6 also show a surprising inhibition of growth of pathogenic bacteria in conditions where C. acnes is either absent or lacks a glycerol substrate in its growth media. Thus, the observed inhibition of the pathogens may not be fully explained by the role of C. acnes. Further, the combination of glycerol and a chitosan-dextran derivative showed a synergistic, or at least an advantageous and beneficial, interrelationship in inhibiting the growth of pathogenic bacteria.

### EXAMPLES

The invention is further described with reference to the following examples. It will be appreciated that the invention as claimed is not intended to be limited in any way by these examples.

### Example 1—Dissolvable Composition

The dissolvable composition used in Examples 2 and 3 comprises a hydrogel of N-succinyl chitosan polymer cross linked with aldehyde derivatised dextran polymer. The N-succinyl chitosan polymer and aldehyde derivatised dextran polymer were prepared according to the methodology described in WO2009/028965.

### Example 2—Preparation of the Dissolvable Composition

A kit comprising precursors of the dissolvable composition was provided, comprising:
Sealed vial A containing 12 mL sodium phosphate buffer solution containing 40% w/v glycerol
Sealed vial B containing 350 mg aldehyde-derivatised dextran polymer powder
Sealed vial C containing 12 mL N-succinyl chitosan polymer (5% w/v) solution in 0.3% sodium phosphate buffer.
The kit further comprised equipment for preparing the dissolvable composition:
Fluid dispensing connector
Two (2) mixing cannulae
Pliable cannula
Two (2) sterile 20 mL Luer lock syringes
Barrier system for containing the precursors and equipment

To prepare the dissolvable composition for administration to a subject, 11 ml of the contents of sealed vial A (sodium phosphate buffer solution) was drawn up by a first sterile 20 mL Luer lock syringe and transferred to opened vial B. Vial B was capped and agitated for 20 seconds. The AB mixture was drawn up into the first syringe and allowed to stand for 15 minutes to ensure dissolution of the aldehyde derivatised dextran polymer powder.

11 mL of sealed vial C was drawn up into a second sterile 20 mL Luer lock syringe. The first and second syringes were connected together via a syringe-to-syringe connector and the AB mixture and C mixture were combined and mixed by transferring the solution between the first and second syringe at least 6 times. The ABC solution was allowed to stand for at least 15 minutes to allow the solution to set into a hydrogel.

The composition thus prepared comprised aldehyde-derivatised dextran polymer cross linked with N-succinyl chitosan polymer, an aqueous phosphate buffer and approximately 20% v/v glycerol.

The hydrogel, prepared according to the method described above, is useful as a stent or packing material. The hydrogel acts as a space-occupying packing material for use as a stent or packing material for application to surgical wounds, particularly surgical wounds associated with endoscopic sinus surgery (ESS).

The hydrogel forms in situ in two 20 mL syringes following the combination of components A, B and C. Once it has formed a gel, it may be applied to the site of a surgical wound. For surgical wounds associated with ESS, the pliable cannula is particularly advantageous in delivering the hydrogel safely and effectively to the wound.

The pliable wires embedded in the tube allow the pliable cannula to be manually bent and shaped into a particular configuration to allow the cannula to be inserted via the nose (for example) or other body orifice to the site of the surgical wound. The pliability of the cannula thus allows the precise application of the hydrogel to the site of the surgical wound without causing further trauma to the patient.

To apply the hydrogel to a surgical wound associated with ESS, the pliable cannula is attached to the end of the syringe containing the hydrogel. The cannula is manually shaped by the health care worker to allow the cannula to be inserted through the nostril to the site of the surgical wound such that the second opening of the cannula is oriented towards the site of the wound. A layer of the hydrogel is then applied to the wound.

The plastic tube itself is a soft plastic material which is sufficiently soft that it does not cause trauma to the patient during insertion of the cannula or application of the hydrogel.

The composition is useful in supporting a healthy skin microbiome when applied to an area of damaged skin, infected skin, or skin in which the microbiome has been disrupted. The kit may therefore be adapted for topical administration of the composition to skin that is damaged, infected or skin that has a disrupted microbiome.

### Example 3—Double-Blinded, Randomised Control Study

Patients over 18 years of age with CRS undergoing primary bilateral full house functional endoscopic sinus surgery (FH-FESS) participated in this trial. Patients with a shell-fish allergy, pregnancy or breast feeding were excluded from the trial.

### Study Design

All participants gave informed consent. Prior to surgery, demographic information was collected, and patients completed a visual analogue scale (VAS) to assess severity of sinonasal symptoms on each side of the sinonasal tract.

All patients underwent primary bilateral FH-FESS performed with cold steel and powered instruments with particular attention given to conserving mucosa. Microbiology and microbiome swabs were taken by the surgeon from the middle meatus on each side of the sinonasal tract under endoscopic guidance at the beginning of surgery. Baseline scoring of the appearance of the sinuses on either side was performed assessing for adhesions, evidence of infection, oedema, crusting and granulation tissue. At the end of surgery, the frontal, sphenoidal and maxillary sinus ostial areas were determined by measuring width and height of the sinus ostia with a standardised 5 mm measuring probe.

Randomisation was performed using GraphPad Quickcalcs software. At the end of the operation, the surgeon was informed which side the patient had randomly been assigned the dissolvable composition prepared according to the method described in Example 2. The dissolvable composition was applied to one side of the sinus and the other side of the sinus was not treated with the dissolvable composition and used as a control.

Up to 20 mL of the composition prepared according to Example 2 was applied using a supplied malleable cannula at the end of surgery under endoscopic guidance filling the frontoethmoidal recess, sphenoid and maxillary sinuses. The nasal tract was not filled with gel to allow for an unobstructed nasal airway. Patients were blinded to which side received treatment. Given that the nasal cavity was not packed and there was no nasal obstruction, patients could furthermore not tell which side received treatment.

Patients received standard post-operative care and follow-up at 2 weeks, 6 weeks and 3 months post-operatively. Following surgery patients received an empirical course of oral antibiotics and were directed to commence 240 mL saline nasal douches bilaterally four times a day, starting the day after surgery. By two weeks post-operative, there was no remaining dissolvable composition in the sinus cavity. At each visit patients had endoscopic debridement of the sinuses as required. Topical steroid, budesonide 1 mg/2 mL (Pulmicort respules 1 mg/2 mL), added to one of the daily saline nasal douches were commenced at two weeks post-operatively. Any infection that developed during follow-up was treated as per standard care with culture directed antibiotics.

At each visit patients completed a VAS to assess severity of sinonasal symptoms on each side of the sinonasal tract. Nasoendoscopies performed at each follow-up were recorded and then assessed by a blinded assessor for adhesions, evidence of infection, oedema, crusting and granulation tissue. Adhesions were assessed by determining the percentage of the height of the head of the middle turbinate taken up by adhesion.

At 3 months post-operatively, microbiology and microbiome swabs were again taken from bilateral middle meati under endoscopic guidance. Sinus ostia were remeasured at this visit using a standardised 5 mm measuring probe. Patients then exited the trial.

### Microbiome Collection and DNA Extraction

Standardised collection of microbiome samples was performed intraoperatively and at 3-months post-operative. One guarded Copan Flocked swab (COPAN ITALIA, Brescia, Italy) was collected from the middle meatus on each side under endoscopic guidance. Swab tips were removed and stored in individual sterile cryotubes which were transported on ice and stored at −80° C.

DNA Extractions were performed as per manufacturer's instructions using the Qiagen DNeasy Blood and Tissue Kit (Qiagen). This was performed under strict sterile conditions using a class 2 biological safety cabinet with new equipment between samples to avoid contamination. With each batch of DNA extraction, a negative control (sterile Copan Flocked swab tip) was used to assess for contamination.

Copan Flocked swabs were cut into 5 mm pieces and placed in a microcentrifuge tube containing 180 μL lysozyme solution (Sigma) at 20 mg/mL in lysis buffer (20 mmol/L Tris-Cl, pH8; 2 mmol/L sodium EDTA; 1.2% Triton X-100, filter-sterilized; Sigma, St Louis, USA). This was left at room temperature overnight. The following day, 5 mm steel beads were added to each microcentrifuge tubes and this was beat with a Tissue Lyser II (Qiagen) at 15 Hz for 20 seconds. The steel beads were removed, and 50 mg of glass beads were added to the microcentrifuge tubes containing the swabs and lysis buffer. This was beat again with a Tissue Lyser at 30 Hz for 5 minutes. 25 μL proteinase K and 200 μL Buffer AL (Qiagen) were added to each microcentrifuge tube and mixed by vortex. These were incubated at 56° C. for 30 minutes. The samples were then centrifuged briefly to separate out the glass beads and the supernatant. The supernatant was transferred to new microcentrifuge tubes and 200 μL 96-100% ethanol was added and mix via vortex. This mixture was pipetted into a DNeasy Mini Spin column (Qiagen). Final elution was performed as per instructions from manufacturer resulting in a total of 100 μL of DNA from each sample. A NanoDrop 1000 Spectrophotometer (Thermo Scientific) was used to establish the concentration of each sample. Samples were stored at −80° C. and transported on ice for sequencing at the Australian Genome Research Facility (AGRF).

### Microbiome Sequencing and Analysis

Bacterial samples underwent polymerase chain reaction (PCR) and sequencing at AGRF. Libraries were generated by amplifying the 341F primer against the V3-V4 hypervariable region of the 16S rRNA gene (CCTAYGGGRBGCASCAG forward primer; GGACTACNNGGGTATCTAAT reverse primer). Sequencing was performed using the Illumina MiSeq platform (illumine Inc).

### Microbiological Samples

One swab (Sigma Transwab®, MWE Medical Wire, UK) was collected from each side of the middle meatus intraoperatively and at 3 months postoperatively under endoscopic guidance. Samples were sent for microscopy, culture and sensitivity to a pathology lab (Clinpath Pathology, Australia). Growth on culture was quantified as ‘scant’, ‘light’, ‘moderate’ or ‘heavy’.

### Statistical Analysis

A mixed linear effects model was used to establish difference in endoscopic scores. A Fisher's exact test was used to analyse differences in rates of infection between the treatment side and control side.

### Results

### Patient Cohort

Twenty patients were recruited. All but one patient were followed up for a minimum of 3 months post-operatively. Ages of the patients ranged from 22 to 75 years of age. There were eight females and twelve males recruited. Nine patients had CRS with nasal polyps and eleven patients had CRS without nasal polyps. Ten patients received the treatment on the left and the other ten on the right. There were no adverse outcomes following use of the treatment and it was extremely well tolerated.

### Symptomatic Scores

Patients graded symptoms of facial pain/discomfort, bleeding, nasal obstruction, nasal secretions, post-nasal drip and sense of smell on a Likert scale, the Visual Analogue Scale (VAS). All patients reported improvement in symptoms following surgery. No significant difference was noted between sides for symptoms of facial pain/discomfort, bleeding, nasal obstruction, nasal secretions, post-nasal drip and sense of smell but there appeared to be a trend for improved symptoms on the treatment side.

### Endoscopic Appearance

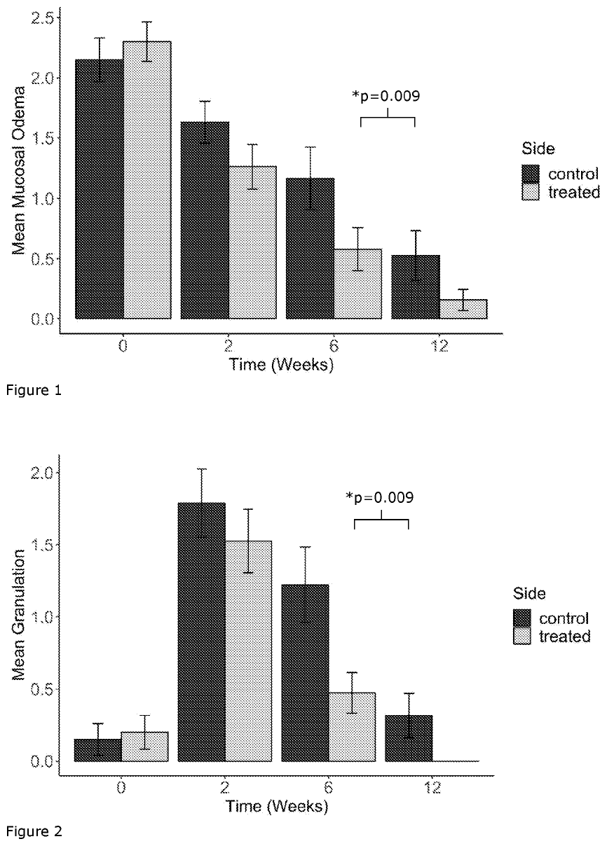

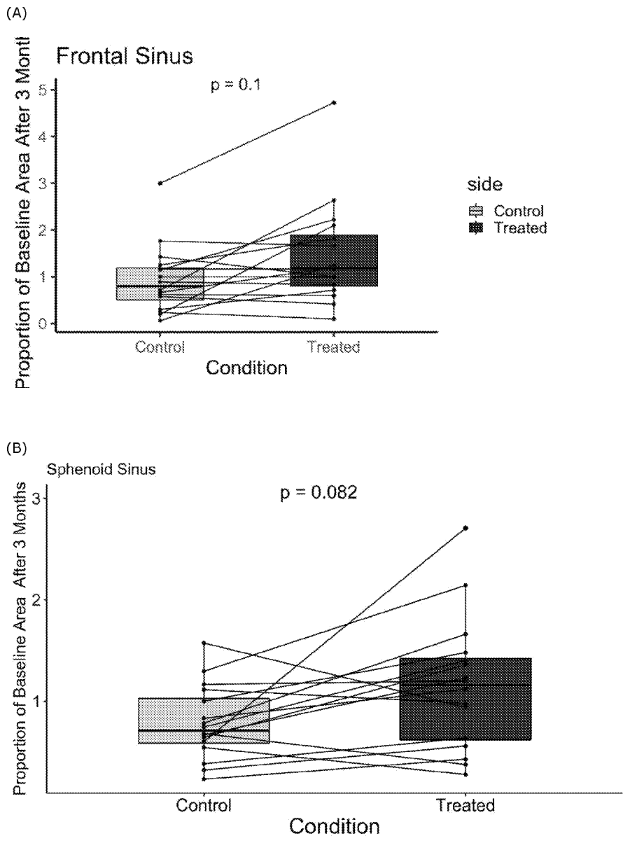

A mixed linear effects model performed on endoscopic scores obtained by a blinded assessor showed a significant decrease in oedema and granulation tissue noted on the treated side compared to control at 6 weeks (p=0.014, and p=0.009 respectively). See FIGS. 1 and 2.

### Ostial Measurements

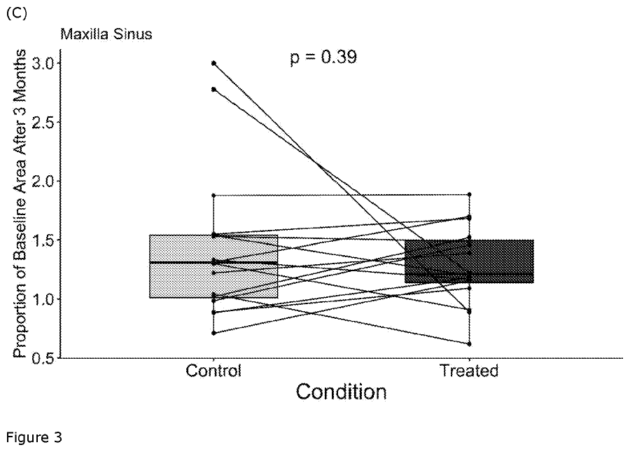

The proportion of ostial area maintained after 3 months was compared between control and treated sides. There was a trend for increased proportion of baseline ostial area maintained at 3 months in the treated side for the frontal and sphenoidal sinuses compared to control. The proportion of baseline maxillary ostial area maintained at 3 months was similar in both treated and control groups. See FIG. 3.

### Rates of Infection

An infection was defined as a score of at least mild mucopurulent discharge on review of endoscopy (as assessed by a blinded reviewer) in conjunction with at least a moderate growth of pathogenic bacteria on microbiology swab taken from the middle meatus.

In the three-month follow-up period, ten patients developed infections, three patients with bilateral infections and seven patients with unilateral infections, which were all noted to be on the control side only. No patient developed a unilateral infection on the treated side. See Table 1.

Two patients had bilateral infections at two weeks, the remaining eight patients had infections at 6-12 weeks. Five patients had infection with P. aeruginosa, four with S. aureus (one of which was multi-resistant) and one patient had an infection caused by Klebsiella aerogenes.

A Fisher test was performed showed a significant decrease in proportion of post-operative infections on the treated side 23% compared to control side 77% (p=0.04).

TABLE 1
Frequency of infections, location of infection and causative
bacterial pathogen at 2, 6 and 12 weeks post-operatively

| Post-operative | Number of | | Causative bacterial |
|---|---|---|---|
| period | infections | Side | pathogen |
| 2 weeks | 2 | Bilateral | S. aureus |
| | | Bilateral | S. aureus |
| 6 weeks | 4 | Control | MRSA |
| | | Control | S. aureus |
| | | Control | P. aeruginosa |
| | | Bilateral | P. aeruginosa |
| 12 weeks | 4 | Control | Klebsiella aerogenes |
| | | Control | P. aeruginosa |
| | | Control | P. aeruginosa |
| | | Control | P. aeruginosa |

### Microbiome

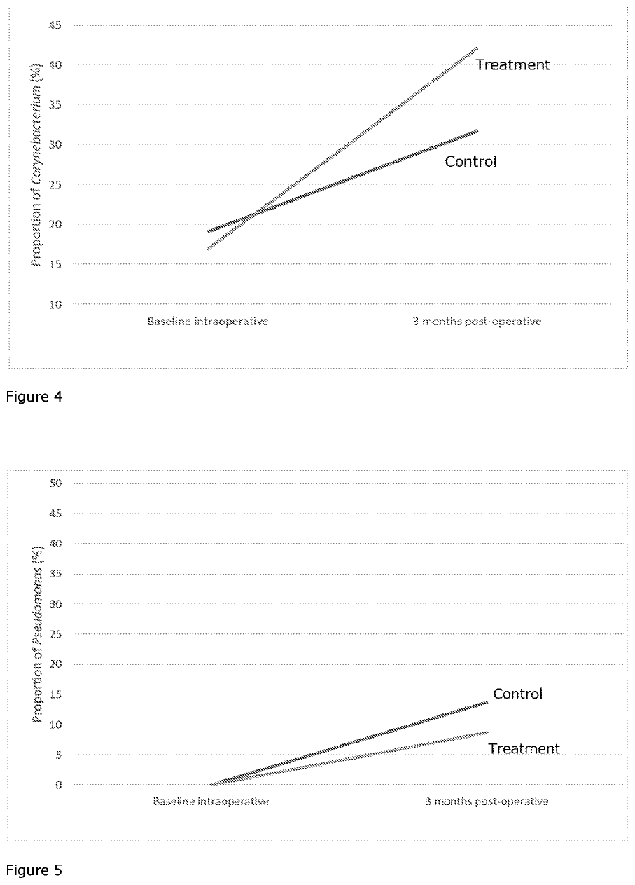

There was an increase in Corynebacterium at 3 months post-operatively in both groups but higher in the treatment group. The proportion of the microbiome that consisted of Corynebacterium increased from 19.1% to 31.7% in the control group. However, the increase in the treated group was higher—17.0% to 42.1%. See FIG. 4.

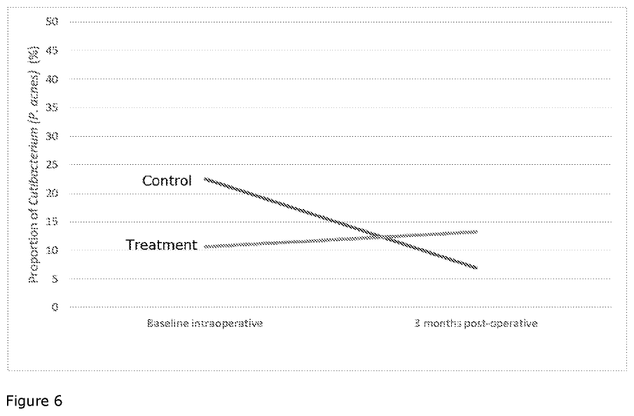

No Pseudomonas was present at the time of operation in either group. At three months post-operatively there is less of an increase in Pseudomonas in the treated group. The proportion of the microbiome that consisted of Pseudomonas at 3 months post-operatively is 8.7% in treated group compared to 13.7% in the control group. See FIG. 5.

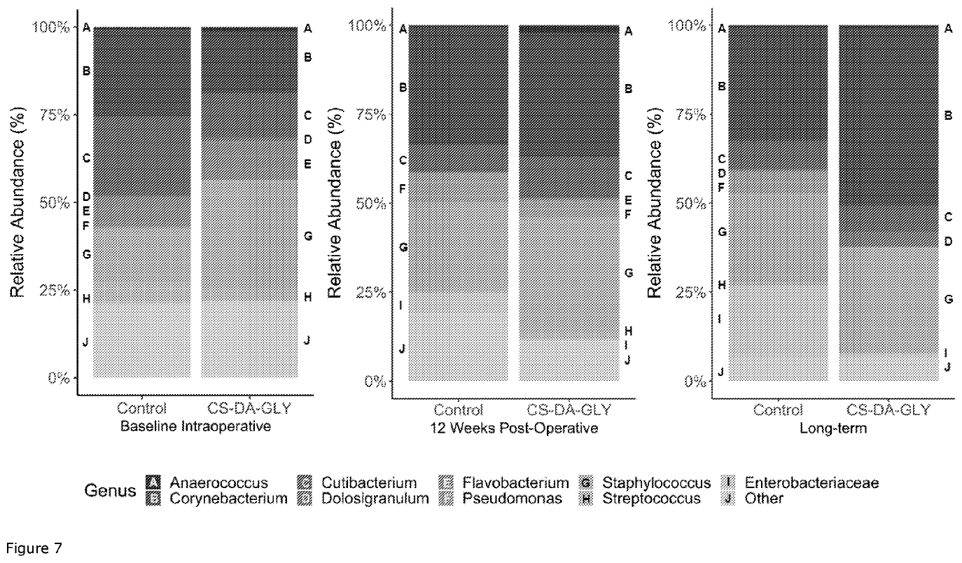

Cutibacteria or C. acnes stayed constant or increased slightly and increased in the treatment group but decreased in the control group at 3 months post-operatively. The proportion of the microbiome that consisted of Cutibacterium decreased from 22.5% to 6.9% in the control group. However, in the treatment group, it actually increased from 10.6% to 13.2%. See FIG. 6.

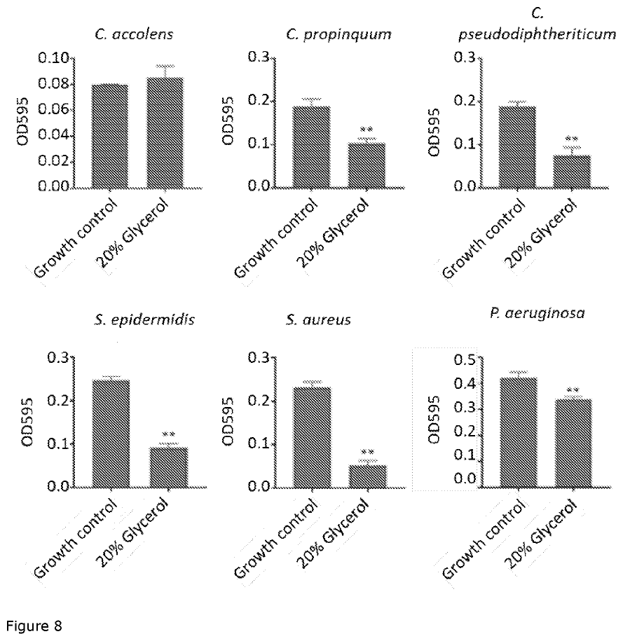

The microbiome profiles for both control and treatment groups at time of operation and 3 months post-operative are shown in FIG. 7. Treatment led to a significant increase in the mean relative abundance of bacteria in the Corynebacterium genus and combined Corynebacterium and Cutibacterium (C. acnes) forming the microbiome, compared to control, and this trend persisted at the long term timepoint (after 12 months).

### Comparison of Microbiome in Patients who Developed Post-Operative Infections

Subgroup analysis was performed to assess the relative abundances of Corynebacterium, C. acnes, Psuedomonas and S. aureus in the ten patients who developed infections in the 3-month post-operative time period compared to the general cohort of patients without infection. Further analysis was then performed to assess the difference in microbiome for the four patients that had infections at the time of DNA collection compared to the general cohort without infection. The relative abundances of Corynebacterium, C. acnes and Pseudomonas, are shown in Table 2.

TABLE 2
Relative abundance of microbiota at the time of surgery compared
to 3 months post-surgery in treatment and control groups.

| | Mean Relative Abundance (%) | | | |
|---|---|---|---|---|
| | | Control 3 | | CD Gel 3 |
| | Control | months post- | CD Gel | months post- |
| Genera | Baseline | operative | Baseline | operative |
| Corynebacterium | 19.1 | 31.7 | 17.0 | 42.1 |
| Cutibacterium | 22.5 | 6.9 | 10.6 | 13.2 |
| (C. acnes) | | | | |
| Pseudomonas | 0 | 13.7 | 0 | 8.7 |

### Example 4—Effect of Glycerol on Growth of Nasal Bacterial Isolates

The effect of glycerol on bacterial growth was determined by analysis of nasal bacterial isolates from subjects participating in the trial described in Example 3. The bacterial isolates were cultured for 24 hours in Nutrient Broth (NB) and Brain-Heart Infusion (BHI) which further contained glycerol in an amount of either 20% v/v, or 40% v/v, or 0% glycerol (control).

A total of 22 nasal clinical isolates including C. accolens (n=4), C. propinquum(n=3), C. pseudodiptheriticum (n=3), S. epidermidis (n=4), S. aureus (n=4) and P. aeruginosa (n=4) were used in this experiment. Prior to starting experiments, isolates were grown in tryptic soya agar (TSA) and incubated at 37° C. under aerobic conditions for 24 hours, except for C. accolens which was incubated for 48 hours.

Glycerol at 20% was prepared by dilution in nutrient broth (NB) media. Glycerol treatments at 20% concentrations were prepared in a 96-well microtitration plate containing 50 μL NB. A single colony of bacterial isolates such as C. accolens, C. propinquum, C. pseudodiptheriticum, S. epidermidis, S. aureus and P. aeruginosa from overnight cultures was suspended with 0.9% saline (aqueous NaCl) to McFarland standard of 0.5 and diluted 1:100 in NB growth media. Next, 50 μL of the diluted bacterial suspension was then added to the treatments in each well and incubated aerobically for 24 hours at 37° C. A negative control (media without bacteria and treatment) and untreated growth control (media with bacteria but no treatment) were used in this assay. After incubation, the planktonic bacterial growth was determined by measuring the optical density (OD) at 595 nm using microplate absorbance reader (iMark™, BIO-RAD). For each bacterial strain, six replicate experiments were performed to assess bacterial growth.

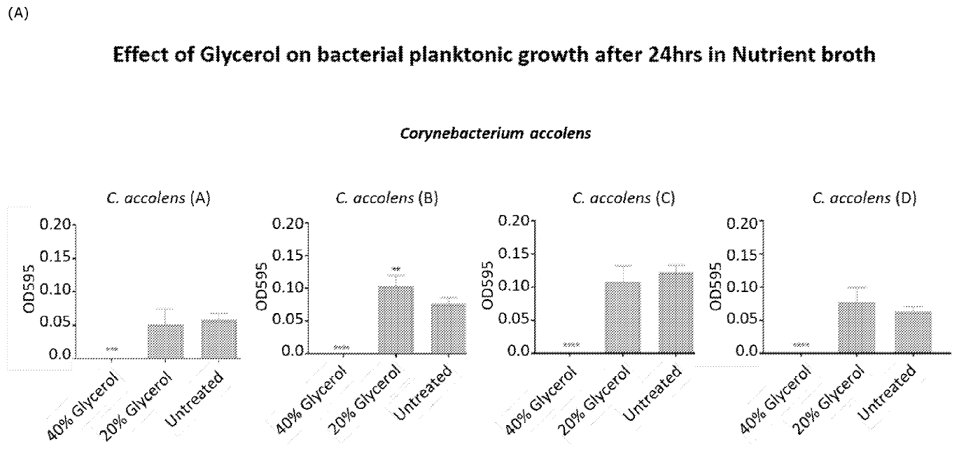

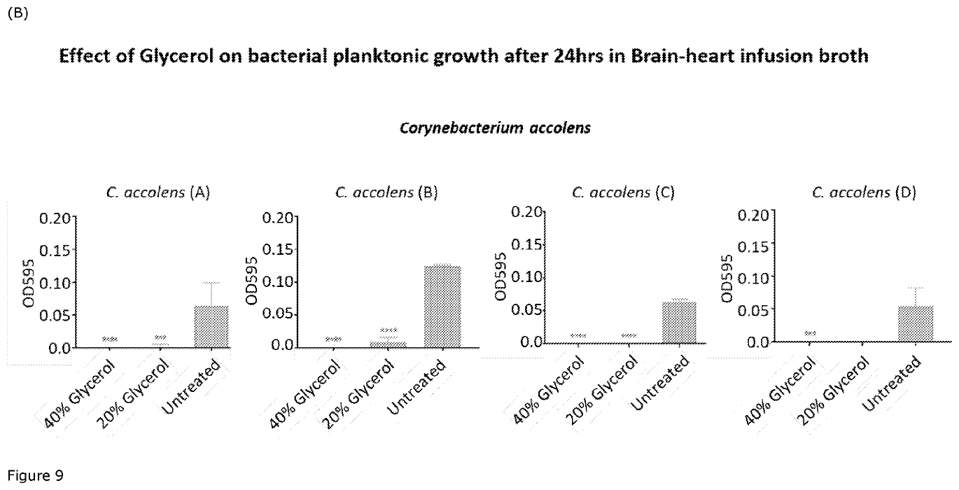

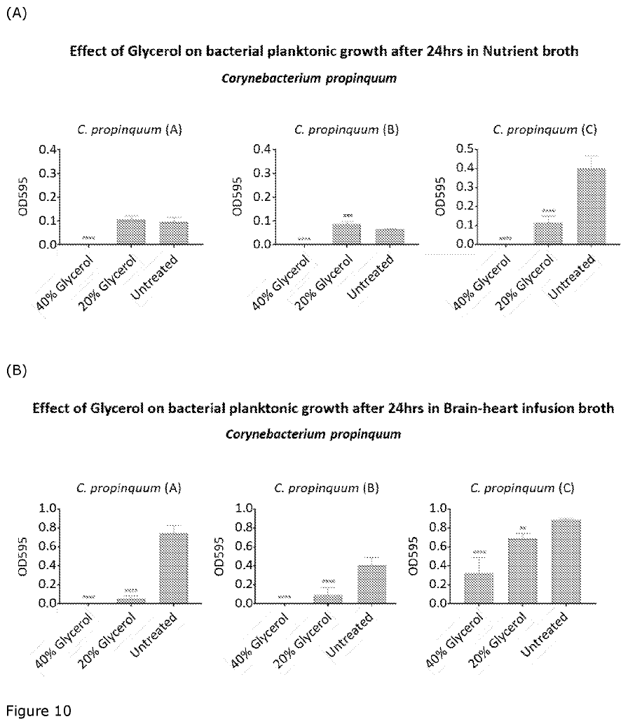

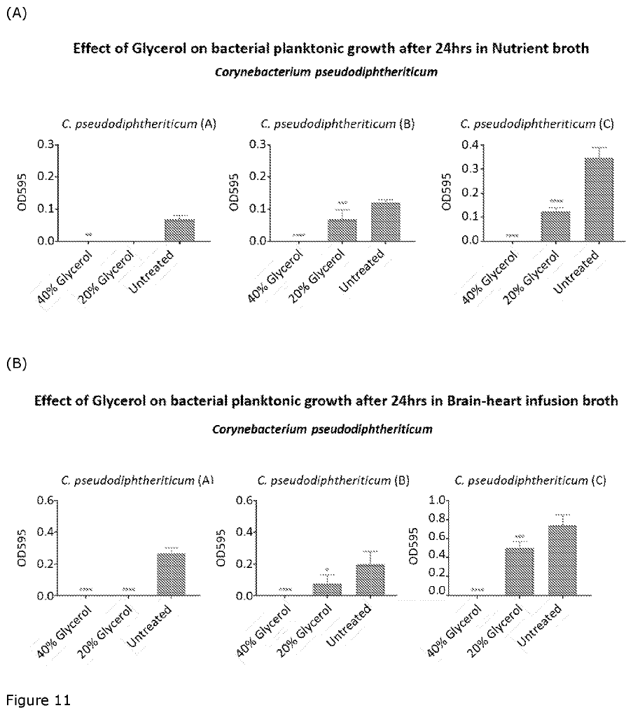

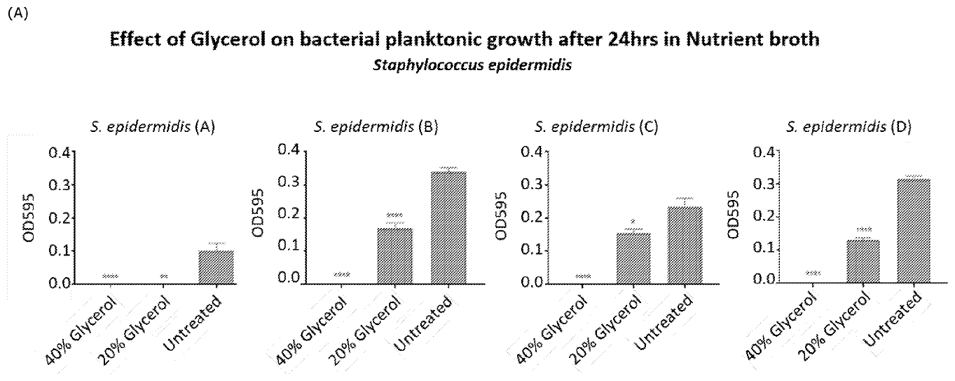

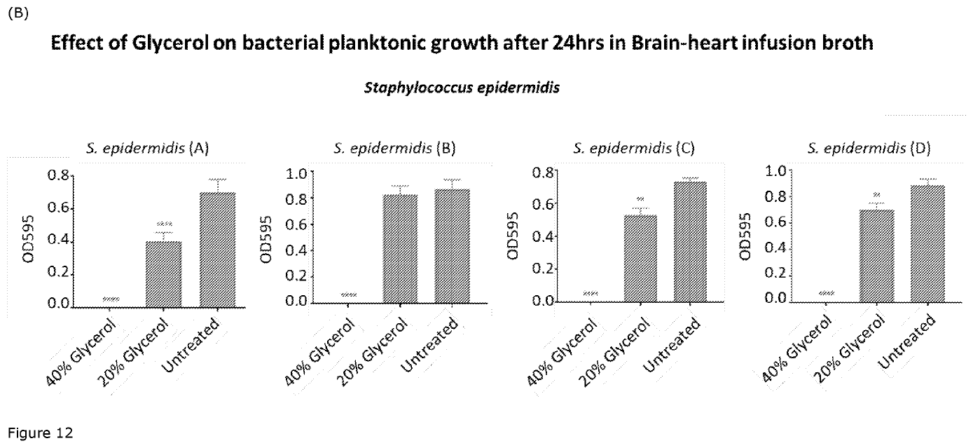

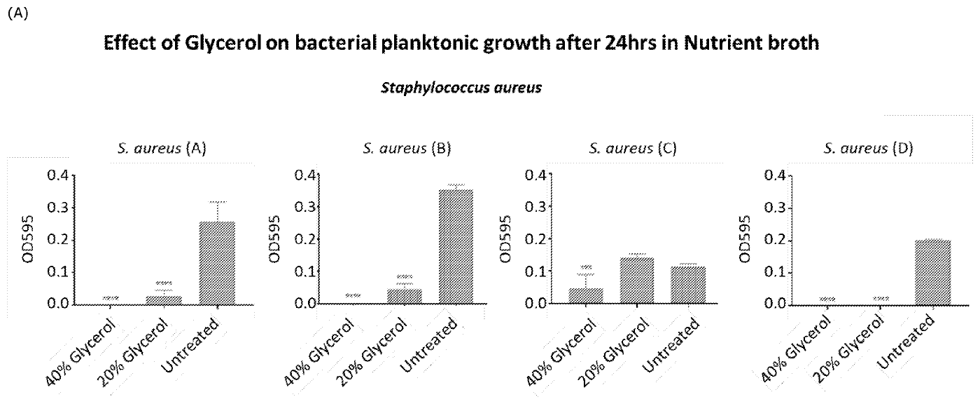

FIGS. 8 to 14 show the growth of bacterial isolates in NB and BHI in the presence of 0%, 20% and 40% (v/v) glycerol.

BHI broth samples comprising 40% and 20% glycerol were found to significantly inhibit the growth of commensal and pathogenic bacterial isolates after 24 hours incubation.

NB samples comprising 40% glycerol were found to significantly inhibit the growth of commensal and pathogenic bacterial isolates after 24 hours incubation. NB samples comprising 20% glycerol were found to promote the growth of C. accolens and C. propinquum after 24 hours incubation, but significantly inhibit the growth of C. pseudodiphtheriticum, S. epidermidis, S. aureus and P. aeruginosa clinical isolates. Glycerol at 20% v/v concentrations significantly reduced the growth of pathogenic bacteria C. propinquum, C. pseudodiptheriticum, S. epidermidis, S. aureus and P. aeruginosa(p<0.05) in a nutrient poor environment, compared to untreated growth controls. However, 20% glycerol did not impact the growth of the nasal commensal bacteria, C. accolens (p>0.05), compared to untreated growth controls.

### Example 5—C. acnes

The growth of C. acnes in the presence of glycerol, chitosan-dextran polymer (CS-DA), and a combination of glycerol and chitosan-dextran polymer (CS-DA-GLY) was investigated according to the following methodology:
Cellulose disks (approximately 1 cm in diameter) pre-soaked in C. acnes culture were placed on the surface of sterile nutrient agar plates. A test substance was placed on top of the soaked disks to allow contact with both the nutrient agar and a test substance. An untreated control agar plate contained only the soaked cellulose disk, with no test substance applied. The test substances were: (1) 20% glycerol in water; (2) a composition comprising aldehyde-derivatised dextran polymer cross linked with N-succinyl chitosan polymer, an aqueous phosphate buffer and approximately 20% v/v glycerol, prepared according to the description above in Example 2 (herein referred to as CS-DA-GLY); and (3) a composition comprising aldehyde-derivatised dextran polymer cross linked with N-succinyl chitosan polymer, and an aqueous phosphate buffer (herein referred to as CS-DA). That is, the composition of test substances (2) and (3) differ only in the presence and absence of glycerol. All test and control cellulose disk plates were incubated for 48±1 hours at conditions appropriate for the microorganism. Following incubation, the cellulose disks were each aseptically removed from the agar surface and the microorganism contents of the cellulose disks were individually extracted using sterile phosphate-buffered saline solution. Each of the resulting microorganism suspension was serially diluted 1:10 in sterile phosphate-buffered saline solution and 1.000 mL of the appropriate dilutions were plated in singlet to sterile growth media appropriate for the target test microorganism. Following incubation for 112 hours 5 minutes at conditions appropriate for the microorganism, the surviving test microorganisms were recorded for each test and control plate.

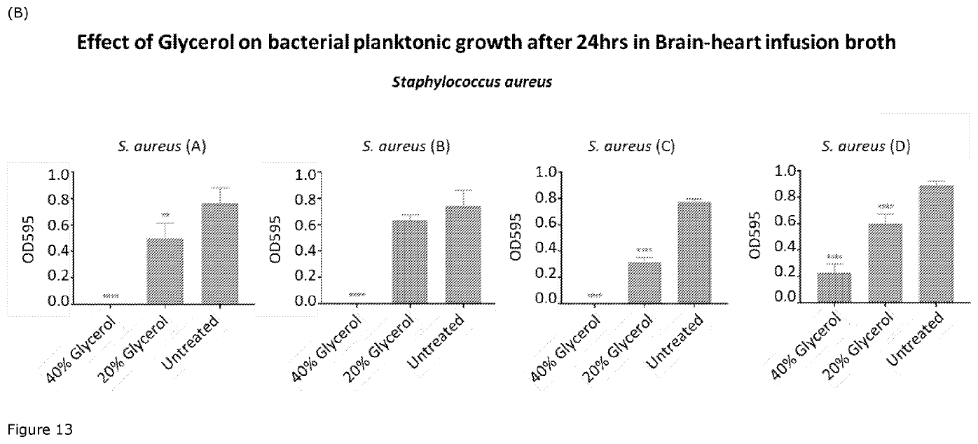

FIGS. 15 shows the effect of glycerol, CS-DA, and CS-DA-GLY on the growth of C. acnes. Growth was supported in the presence of glycerol and CS-DA-GLY but appeared to be less supported in the presence of CS-DA, compared to untreated control.

### Example 6—S. aureus and P. aeruginosa

The growth of S. aureus and P. aeruginosa in the presence of glycerol, chitosan-dextran polymer, or a combination of glycerol and chitosan-dextran polymer, was investigated according to the following methodology:
(A) Cellulose disks (approximately 1 cm in diameter) pre-soaked in S. aureus or P. aeruginosa cultures were placed on the surface of sterile nutrient agar plates. A test substance was placed on top of the soaked disks to allow contact with both the nutrient agar and a test substance. An untreated control agar plate contained only the soaked cellulose disk, with no test substance applied. The test substances were as described in Example 5, namely: (1) 20% v/v glycerol in water; (2) CS-DA-GLY; and (3) CS-DA. All test and control cellulose disk plates were incubated for 48±1 hours at conditions appropriate for each microorganism (e.g. aerobic conditions for S. aureus and P. aeruginosa, and anaerobic conditions for C. acnes). Following incubation, the cellulose disks were each aseptically removed from the agar surface and the microorganism contents of the cellulose disks were individually extracted using sterile phosphate-buffered saline solution. Each of the resulting microorganism suspensions were serially diluted 1:10 in sterile phosphate-buffered saline solution and 1.000 mL of the appropriate dilutions were plated in singlet to sterile growth media appropriate for the target test microorganism. Following incubation for 72 hours 40 minutes at conditions appropriate for each microorganism, the surviving test microorganisms were recorded for each test and control plate.
(B) The same methodology as (A) was performed, but the cellulose disk plates additionally included the supernatant of a C. acnes culture that was grown on a substrate comprising 20% glycerol. C. acnes supernatant was spread on the agar surface, and also half of the supernatant of S. aureus and P. aeruginosa was replaced with supernatant from C. acnes.
(C) The same methodology as (A) was performed, but the cellulose disk plates additionally included the supernatant of a C. acnes culture that was grown on a glycerol-free substrate. C. acnes supernatant was spread on the agar surface, and also half of the supernatant of S. aureus and P. aeruginosa was replaced with supernatant from C. acnes.
   S. aureus

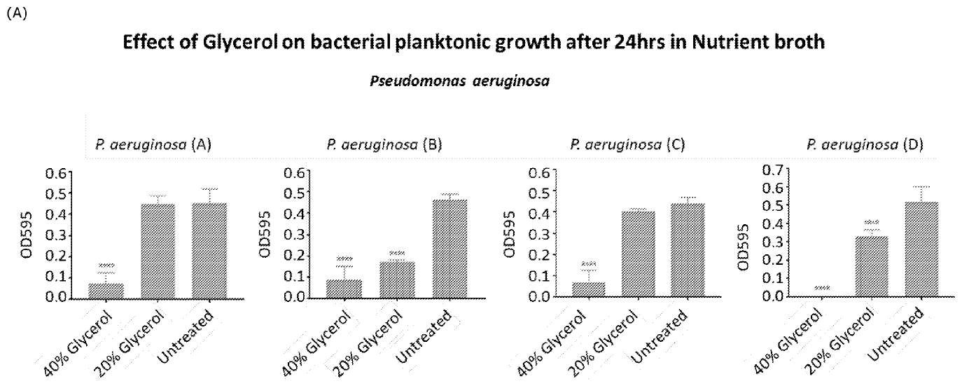

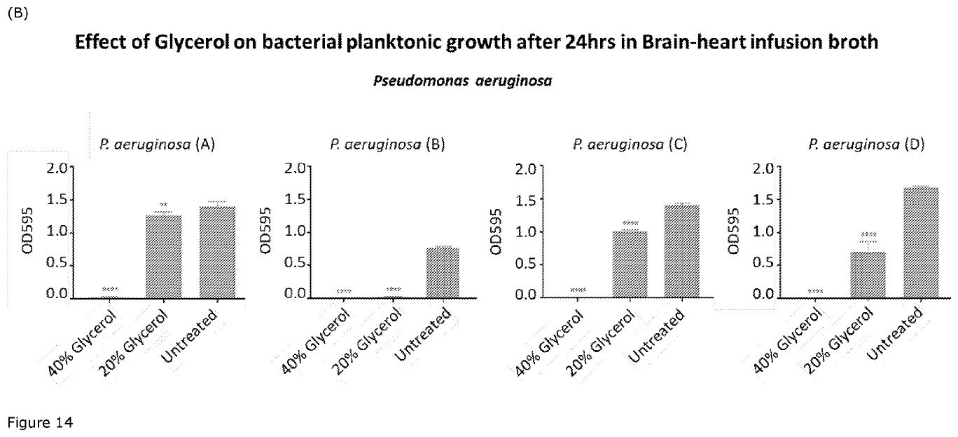

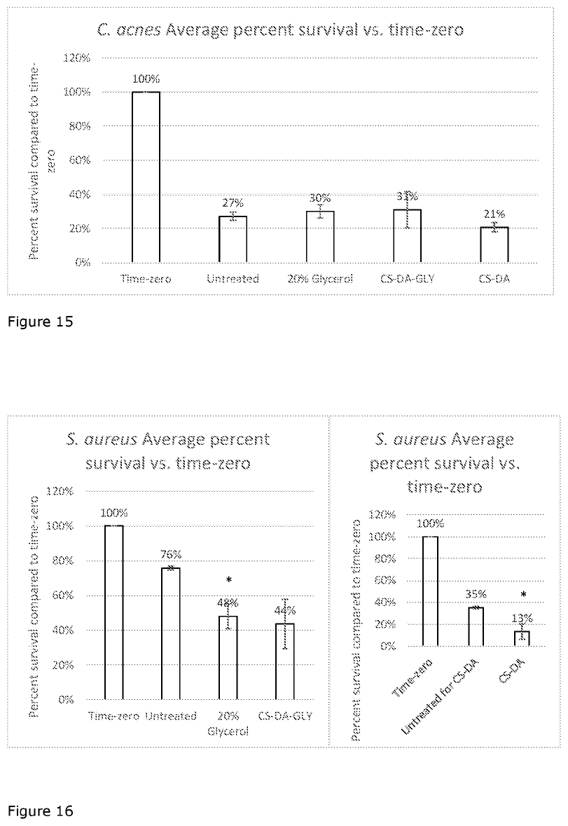

FIGS. 16, 17, and 18 show the effect of glycerol, CS-DA, and CS-DA-GLY on the growth of S. aureus. In FIGS. 17 and 18, the “Untreated” samples still comprise the C. acnes supernatant but are untreated in that no test substance was applied (glycerol/CS-DA/CS-DA-GLY).

In test set A (i.e., no C. acnes supernatant), the application of glycerol, CS-DA-GLY, and CS-DA caused a decrease in the growth of S. aureus compared to control. In test sets B and C (i.e., comprising C. acnes supernatant), only the application of glycerol and CS-DA-GLY caused a decrease in the growth of S. aureus compared to control. In test sets B and C, CS-DA caused an increase in the growth of S. aureus compared to control.

The presence of culture supernatant from a C. acnes culture grown in the presence of glycerol (i.e., test set C) in the cellulose disk plates caused a substantial decrease in growth of S. aureus, regardless of the presence or type of test substance.

There is a synergistic effect between the combination of C. acnes supernatant and the treatment with either 20% glycerol or CS-DA-GLY, which is not observed in groups comprising CS-DA. This indicates that the glycerol is the component responsible for this synergy.

P. aeruginosa

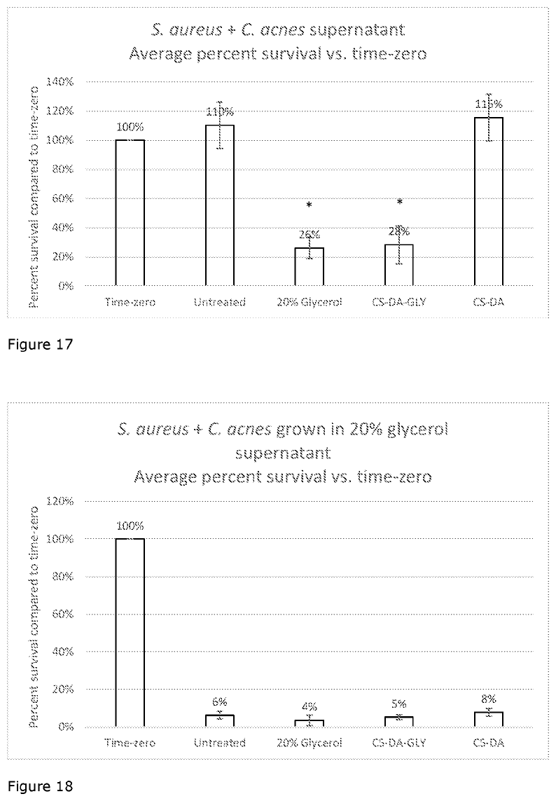

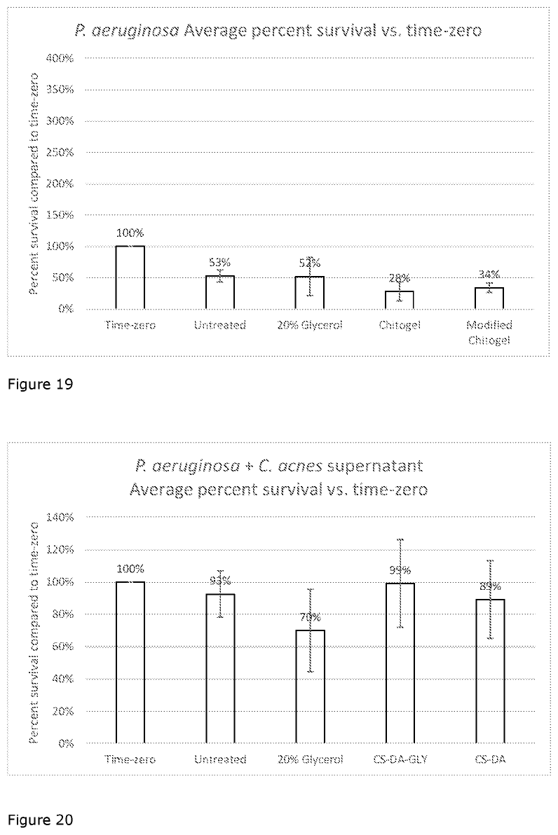

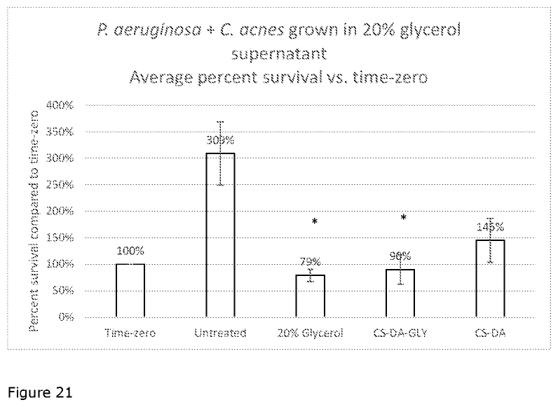

FIGS. 19, 20, and 21 show the effect of glycerol, CS-DA, and CS-DA-GLY on the growth of P. aeruginosa. In FIGS. 20 and 21, the “Untreated” samples still comprise the C. acnes supernatant but are untreated in that no test substance was applied (glycerol/CS-DA/CS-DA-GLY).

In test set A, the application of glycerol, CS-DA-GLY, and CS-DA caused a decrease in the growth of P. aeruginosa compared to control. Test set C showed a much more substantial decrease in the growth of P. aeruginosa for glycerol, CS-DA-GLY and CS-DA compared to control. Particularly notably, test set C showed a substantial growth of P. aeruginosa in the untreated control, as well as growth in the presence of CS-DA, but showed a reduction in the presence of glycerol and CS-DA-GLY. This suggests that the combination of C. acnes supernatant grown in the presence of glycerol, and either glycerol or CS-DA-GLY have a synergistic effect on the reduction or inhibition of growth of P. aeruginosa.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

1. A method for treating, preventing, reducing the likelihood of, or reducing the severity of, an infection in the sinonasal tract, comprising the topical administration of a composition comprising glycerol to a surface of the sinonasal tract in a subject in need thereof.

2. The method of claim 1, wherein the composition comprises a hydrogel.

3. The method of claim 1, wherein the composition comprises a polymer comprising chitosan.

4. The method of claim 1, wherein the composition comprises a dicarboxy-derivatised chitosan polymer cross-linked to an aldehyde-derivatised dextran polymer.

5. The method of claim 1, wherein the composition comprises at least 20% glycerol (v/v).

6. The method of claim 1, wherein the composition is sterile, and the method is performed without the intentional application or addition of any microorganisms.

7. The method of claim 1, wherein the infection is a post-operative infection in the sinonasal tract.

8. The method of claim 1, wherein the composition is applied to a surface of the sinonasal tract at or around the site of a surgical wound.

9. The method of claim 1, wherein the infection is a Staphylococcus aureus or Pseudomonas aeruginosa infection.

10. The method of claim 1, wherein the infection is associated with chronic rhinosinusitus.

11. A method for promoting the growth of one or more commensal bacteria in the sinonasal tract, comprising the topical administration a composition comprising glycerol to a surface of the sinonasal tract in a subject in need thereof.

12. The method of claim 11, wherein the commensal bacteria are selected from the group consisting of: Cutibacterium acnes and bacteria of the Corynebacterium genus.

13. The method of claim 11, wherein the composition comprises a hydrogel.

14. The method of claim 11, wherein the composition comprises a polymer comprising chitosan.

15. The method of claim 11, wherein the composition comprises a dicarboxy-derivatised chitosan polymer cross-linked to an aldehyde-derivatised dextran polymer.

16. The method of claim 11, wherein the composition comprises at least 20% glycerol (v/v).

17. The method of claim 11, wherein the composition is sterile, and the method is performed without the intentional application or addition of any microorganisms.

18. The method of claim 11, wherein the composition is applied to a surface of the sinonasal tract at or around the site of a surgical wound.

19.-27. (canceled)
